(19) United States
(12) Patent Application Publication
(10) Pub. No.: US 2015/0338422 A1
(43) Pub. Date: Nov. 26, 2015

(54) METHOD FOR OBTAINING DATA THAT ARE USEFUL FOR THE DIAGNOSIS, PROGNOSIS AND CLASSIFICATION OF PATIENTS WITH CHRONIC OBSTRUCTIVE PULMONARY DISEASE (COPD) AND/OR LUNG CANCER

(71) Applicants: SERVICIO ANDALUZ DE SALUD, Sevilla (ES); CONSEJO SUPERIOR DE INVESTIGACIONES CIENTÍFICAS (CSIC), Sevilla (ES); UNIVERSIDAD DE SEVILLA, Sevilla (ES)

(72) Inventors: Luis Gonzalo Paz-Ares Gonzaga, Sevilla (ES); María Dolores Pastor Herrera, Sevilla (ES); Sonia Molina Pinelo, Sevilla (ES); Amancio Carnero Moya, Sevilla (ES); Ana Salinas, Sevilla (ES); Ana Barbosa De Souza Nogal, Sevilla (ES)

(21) Appl. No.: 14/405,685

(22) PCT Filed: Jun. 5, 2013
(86) PCT No.: PCT/ES2013/070364

§ 371 Date: Jul. 30, 2015

(30) Foreign Application Priority Data
Jun. 5, 2012 (ES) P201230874

(51) Int. Cl. G01N 33/74; G01N 33/68; C01B 21/092; G01N 33/574

(52) U.S. Cl. CPC G01N 33/74; G01N 33/57488; G01N 33/57423; G01N 33/5743; G01N 33/57492; G01N 33/6869; G01N 33/6893; G01N 33/6863; C01B 21/092; G01N 2800/122; G01N 2800/52; G01N 2800/50

(57) ABSTRACT

The invention describes a method for obtaining data useful for the diagnosis, prognosis and classification of individuals with chronic obstructive pulmonary disease (COPD) and/or lung cancer, diagnostic kit, device and uses thereof for the diagnosis, prognosis and classification of patients as a) individuals with no COPD or lung cancer, b) individuals with COPD, c) individuals with adenocarcinoma, d) individuals with COPD and adenocarcinoma, or e) individuals with COPD and squamous carcinoma

This invention is within the field of molecular biology and medicine and relates to a method for obtaining data useful for the diagnosis, prognosis and classification of individuals into:
individuals who neither exhibit COPD nor lung cancer
individuals with COPD
individuals with adenocarcinoma
individuals with squamous carcinoma
individuals with COPD and adenocarcinoma
individuals with COPD and squamous carcinoma

### PRIOR ART

Lung cancer (LC) is the cause most commonly associated with mortality related to cancer all over the world; over 1.3 million deaths every year are attributed to it, it accounts for 12.7% of all new cases of cancer. This malignancy is divided into two major groups according to its clinical-pathological characteristics: small-cell lung carcinoma (SCLC) and non-small cell lung carcinoma (NSCLC). About 75-805% of the cases of LC belong to the group of non-small cell lung cancer. Histologically, non-small cell lung cancer is subdivided into two main categories. Squamous cell lung carcinoma (SCC), also known as epidermoid carcinoma, that generally occurs in the bronchial epithelium. Adenocarcinoma generally occurs in the peripheral airways and the alveoli. The therapeutic options available to date for the treatment of lung cancer (LC) include surgery, chemotherapy and radiation therapy, which can be used separately or in different combinations. However, despite all progress made, the five-year survival rate does not exceed 15% for this type of tumour.

The main risk factor for the development of LC is smoking. About 85-90% of the cases of LC are caused by this habit. On the other hand, smoking is also responsible for developing other diseases, such as chronic obstructive pulmonary disease (COPD). The World Health Organisation (OMS) estimates that three million people die from COPD every year. As with LC, not all smokers develop COPD. However, about 50% of the smokers ultimately experience COPD. In addition, COPD increases the relative risk of lung cancer from two to six times. Several studies suggest that inflammation could be one of the main conditions involved in the pathogenesis of both diseases. The large family of cytokines may be largely mediating this process

Cytokines are a diverse group of proteins including pro-inflammatory cytokines, T-cell derived cytokines, chemotactic cytokines (chemokines) of eosinophils, neutrophils, monocytes/macrophages and T cells, anti-inflammatory cytokines and several growth factors. Several studies performed in patients diagnosed with COPD show that there is an increase in the levels of pro-inflammatory cytokines and chemokines (IL-1β, IL-2, IL-6, IL-8, IL-13, IP-10, INF-γ, MCP-1 and TNF-α) in samples obtained by induction of sputum as compared to healthy controls. In this line, the tumour necrosis factor α (TNF alpha) and soluble TNF receptors increase in the sputum of patients with COPD as compared to healthy smokers.

On the other hand, studies on lung cancer evidence the role of cytokines in this condition, in relation to studies on COPD. For instance, the studies on polymorphisms show that there are specific polymorphism in genes IL-1A and 1B that increase the risk of LC, in particular among subjects of age and with a significant history of smoking.

Despite the studies performed in this field, the results obtained are not sufficient to explain the role played by inflammation in both conditions, as well as the possible mechanisms shared and those independent from each other. With this regard, several inflammatory markers have been analysed both in LC and in COPD independently. However, it is still necessary to find an alternative method of prediction, diagnosis and/or prognosis which allows to sub-classify the individuals with lung cancer and/or chronic obstructive pulmonary disease.

### DESCRIPTION OF THE INVENTION

The authors of this invention have analysed the family members of cytokines and growth factors in bronchoalveolar lavage (BAL) of patients with COPD, adenocarcinoma, squamous carcinoma, with COPD and adenocarcinoma simultaneously, and patients with COPD and squamous cell cancer. In addition, they have validated the results for IL-11 and CCL-1, finding that both could be biomarkers predictive of adenocarcinoma and could improve the early diagnosis of adenocarcinoma of the lung in high-risk smokers, regardless of the presence or absence of COPD.

This invention provides a method for obtaining data useful for the diagnosis, prognosis and classification of individuals with these diseases.

Therefore, a first aspect of the invention relates to the use of cytokines and growth factors selected from IL-6sR, IL-1a, IL-11, CCL-1, EOTAXIN-2, PDGFAA, TNFRI, TNFRII, EGF, MIP-1B, MIG, MCP-1, IGFBP2, IGFBP1, GDF-15, VEGF or any of its combinations, for the prediction, diagnosis, prognosis and classifications of individuals into:

a) individuals with no COPD or lung cancer,
b) individuals with COPD,
c) individuals with adenocarcinoma,
d) individuals with squamous carcinoma,
e) individuals with COPD and adenocarcinoma, or
f) individuals with COPD and squamous carcinoma.

Another aspect of the invention relates to the simultaneous use of cytokines and the growth factors selected from the list consisting of IL-6sR, IL-1a, IL-11, CCL-1, EOTAXIN-2, PDGFAA, TNFRI, TNFRI, EGF, MIP-1B, MIG, MCP-1, IGFBP2, IGFBP1, GDF-15 and VEGF for the prediction, diagnosis, prognosis and classification of individuals into:

a) individuals with no COPD or lung cancer,
b) individuals with COPD,
c) individuals with adenocarcinoma,
d) individuals with squamous carcinoma,
e) individuals with COPD and adenocarcinoma, or
f) individuals with COPD and squamous carcinoma.

A preferred embodiment relates to the use of IGFBP1, MIP1β, CCL-1, MIG, PDGFAA, GDF-15, VEGF and EGF for the prediction, diagnosis, prognosis and classification of individuals into:

a) individuals with no COPD or lung cancer,
b) individuals with COPD,
c) individuals with adenocarcinoma,
d) individuals with squamous carcinoma,
e) individuals with COPD and adenocarcinoma, or
f) individuals with COPD and squamous carcinoma.

From the examples of this invention it is evidenced a correlation between high expression levels of IL-11 with cell proliferation, invasiveness, metastasis and poor prognosis. In addition, IL-11 and CCL-1 evidence statistically significant expression differences in patients with adenocarcinoma versus the other patient groups.

Therefore, another preferred embodiment relates to the use of IL-11 and/or CCL-1 for prediction or prognosis, or for the early diagnosis of adenocarcinoma of the lung in an individual. In a more preferred embodiment, the individual is a smoker.

Another aspect of the invention relates to a method for obtaining useful data, hereinafter referred to as the first method of the invention, for the diagnosis, prognosis and classification of patients in a) individuals with no COPD or lung cancer, b) individuals with COPD, c) individuals with COPD and lung cancer, c) individuals with adenocarcinoma, d) individuals with squamous carcinoma, e) individuals with COPD and adenocarcinoma, or f) individuals with COPD and squamous carcinoma, which comprises:
i) obtaining an isolated biological sample from an individual, and
ii) quantifying the expression product of cytokines and growth factors selected from the list consisting of IL-6sR, IL-1a, IL-11, CCL-1, EOTAXIN-2, PDGFAA, TNFRI, TNFRII, EGF, MIP-1B, MIG, MCP-1, IGFBP2, IGFBP1, GDF-15 and VEGF or any combination thereof.

In a preferred embodiment of this aspect of the invention, the expression of cytokines and growth factors IL-6sR, IL-1a, IL-11, CCL-1, EOTAXIN-2, PDGFAA, TNFRI, TNFRII, EGF, MIP-1B, MIG, MCP-1, IGFBP2, IGFBP1, GDF-15 and VEGF is quantified simultaneously.

In another preferred embodiment, the method of the invention also comprises:
iii) comparing the quantities obtained in step (ii) to a reference quantity.

The reference quantity is obtained from the values of constitutive expression of the genes for cytokines or growth factors, in a group of healthy patients or preferably who do not exhibit COPD or lung cancer.

More preferably, the first method of the invention comprises quantifying simultaneously the expression products of IGFBP1, MIP1β, CCL-1, MIG, PDGFAA, GDF-15, VEGF and EGF.

Steps (ii) and/or (iii) of the methods described above can be completely or partially automated, for instance, by a sensor robotic equipment for the detection of the quantity in step (ii) or the computerised comparison in step (iii).

An “isolated biological sample” includes, but is not limited to, cells, tissues and/or biological fluids of an organism, obtained by any method known to one skilled in the art. Preferably, the biological sample isolated from an individual in step (i) is the washing or the fluid or bronchoalveolar lavage (BAL).

The term “individual” as used in the description, relates to animals, preferably mammals, and more preferably humans. The term “individual” is not intended to be limiting in any aspect, and this can be of any age, sex and physical condition.

The detection of the quantity of IL-6sR, IL-1a, IL-11, CCL-1, EOTAXIN-2, PDGFAA, TNFRI, TNFRII, EGF, MIP-1B, MIG, MCP-1, IGFBP2, IGFBP1, GDF-15 and VEGF can be performed by any means known in the state of the art. The authors of this invention have shown that the detection of the quantity or concentration of antibodies against these cytokines and growth factors semi-quantitatively or quantitatively allow to distinguish the different histological types of lung cancer. Therefore, a differential diagnosis can be established in individuals affected by the abovementioned diseases, which allows to subclassify them.

The measurement of the quantity or concentration of these cytokines and growth factors, preferably in a semi-quantitative or quantitative manner, can be performed directly or indirectly. The direct measurement refers to the measurement of the quantity or concentration of the product of gene expression, based on a signal obtained directly from the transcripts of the genes, based on a signal obtained directly from the transcripts of said genes or of the proteins, and that are related directly to the number of molecules of RNA or proteins produced by the genes. Said signal—to which we can also refer to as intensity signal—can be obtained, for instance, measuring an intensity value of a chemical or physical property of said products. The indirect measurement includes the mean obtained of a secondary component or a biological measurement system (for instance, the measurement of cell responses, ligands, “labels” or enzyme reaction products).

The term “quantity”, as used in the description, refers, though not limited to, to the absolute or relative quantity of the expression products of genes or antibodies, as well as to any other value or parameter related to them or that can be derived from these. These values or parameters comprise values of intensity of signal obtained from any of the physical or chemical properties of these expression products obtained by direct measurement. In addition, said values or parameters include all those obtained by indirect measurement, for instance, any of the measuring systems described in another part of this document.

The term “comparison”, as used in the description, refers, though not limited to, to the comparison of the quantity of the expression products of genes or the quantity of antibodies to IL-6sR, IL-1a, IL-11, CCL-1, EOTAXIN-2, PDGFAA, TNFRI, TNFRII, EGF, MIP-1B, MIG, MCP-1, IGFBP2, IGFBP1, GDF-15 and VEGF of the biological sample to be analysed, also called test biological sample, with an a quantity of the expression products of genes or with a quantity of antibodies to IL-6sR, IL-1a, IL-11, CCL-1, EOTAXIN-2, PDGFAA, TNFRI, TNFRI, EGF, MIP-1B, MIG, MCP-1, IGFBP2, IGFBP1, GDF-15 and VEGF of one or several desired reference samples. The reference sample can be analysed, for instance, simultaneously or consecutively, together with the test biological sample. The comparison described in section (iii) of the method of this invention can be performed manually or computer-aided.

The term “expression product” also called “gene product” refers to the biochemical material, either RNA or protein, result of the expression of a gene. Sometimes a measurement of the quantity of gene product is used to conclude how active a gene is.

The term “reference quantity”, as used in the description, refers to the absolute or relative quantity (to the reference gene) of expression products of genes or antibodies to IL-6sR, IL-1a, IL-11, CCL-1, EOTAXIN-2, PDGFAA, TNFRI, TNFRII, EGF, MIP-1B, MIG, MCP-1, IGFBP2, IGFBP1, GDF-15 and VEGF which allow to distinguish among) individuals with no COPD or lung cancer, b) individuals with COPD, c) individuals with adenocarcinoma, d) individuals with squamous carcinoma, e) individuals with COPD and adenocarcinoma, or f) individuals with COPD and squamous carcinoma.

The adequate reference quantities can be established by the method of this invention from a reference sample that can be analysed, for instance, simultaneously or consecutively, together with the test biological sample. For instance, though not limited to, the reference sample can be the negative controls, i.e., the quantities detected by the invention method in samples of subjects not suffering any of these diseases.

The soluble form of the interleukin 6 (IL-6sR) receptor, with a molecular weight of about 50 k Da has been found in the urine of adult humans (Novick, D. et al. (1989) J. Exp. Med. 170:1409) in culture media conditioned by the growth of the cell line of human myeloma (Nakahima, T. et al. (1992) Jpn. J. Cancer Res. 83:373), in supernatants of culture of PH-stimulated human PBMC and HTLV-1-positive T cell lines (Honda, M. et al. (1992) J. Immunol. 148:2175) and in the serum of HIV-seropositive blood donors (Honda M. et al. (1992) J. Immunol. 148:2175). This solouble form of the receptor apparently arises from the proteolytic rupture of the bond of the IL-6-R membrane. Its amino acid sequence is found with access number in the GenBank (NCBI) NP 000556.1 and/or SEQ ID NO.: 2.

In the context of the present invention, IL-6sR is also defined by a sequence of nucleotides or polynucleotide, which constitutes the coding sequence of the protein contained in SEQ ID NO.: 2, and which would comprise several variants from:

a) nucleic acid molecules encoding a polypeptide comprising the amino acid sequence of SEQ ID NO.: 2,
b) nucleic acid molecules, the complementary chain of which hybridises with the polynucleotide sequence of a),
c) nucleic acid molecules, the sequence of which differs from a) and/or b) due to degeneration of the genetic code,
d) nucleic acid molecules encoding a polypeptide comprising the amino acid sequence with an identity of at least 80%, 90%, 95%, 98% or 99%, with the SEQ ID NO.: 2, and wherein the polypeptide encoded by said nucleic acids has the activity and structural characteristics of the protein IL-6sR. Among said nucleic acid molecules, the one contained in the sequence of the GenBank (NCBI) NM_{—}000565.3o SEQ ID NO.: 1 is included.

The gene IL-1a or interleukin 1, alpha (L-1A, IL1, IL1-ALPHA, IL1F1, IL-1 alpha; hematopoeitin-1; interleukin-1 alpha; preinterleukin 1 alpha; pro-interleukin-1-alpha) is found in chromosome 2 (2q14) and codes for a protein that is member of the family of cytokines interleukin 1. It is a pleitropic cytokine involved in several immune responses, inflammatory conditions and haematopoiesis. This cytokine is produced by monocytes and macrophages to a protein, that is proteolytically processed and released in response to cell damage and induces apoptosis. This gene and 8 other genes of the interleukin 1 family form a genetic cluster in chromosome 2. It has been suggested that the polymorphism of these genes is associated with rheumatoid arthritis and Alzheimer's disease.

In the context of the present invention, IL-1a is also defined by a sequence of nucleotides or polynucleotide, which constitutes the coding sequence of the protein contained in SEQ ID NO.: 4, and which would comprise several variants from:

a) nucleic acid molecules encoding a polypeptide comprising the amino acid sequence of SEQ ID NO.: 4,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 4, wherein the polypeptide encoded by said nucleic acids has the activity and structural characteristics of protein IL-6sR. Among said nucleic acid molecules, the one contained in the sequence of GenBank (NCBI) NM 000575.3 or SEQ ID NO.: 3 is included.

The gene IL-11 or interleukin 11 (AGIF; IL-11) is contained in chromosome 19 (19q13.3-q13.4) encoding for a protein that is a member of the family of cytokines gp130. These cytokines direct the assembly of receptor complexes with multisubunits.

In the context of the present invention, IL-11 is also defined by a sequence of nucleotides or polynucleotide, which constitutes the coding sequence of the protein contained in SEQ ID NO.: 6, and which would comprise several variants from:

a) nucleic acid molecules encoding a polypeptide comprising the amino acid sequence of SEQ ID NO.: 6,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 6, wherein the polypeptide encoded by said nucleic acids has the activity and structural characteristics of protein IL-11. Among said nucleic acid molecules, the one contained in the sequence of GenBank (NCBI) NM 000641.2 or SEQ ID NO.: 5 is included.

The gene CCL-1 or chemokine (C-C motif) ligand 1 (−309, P500, SCYA1, SISe, TCA3, C-C motif chemokine 1; T lymphocyte-secreted protein 1-309; inflammatory cytokine 1-309; small inducible cytokine A1 (I-309, homologous to mouse Tca-3); small-inducible cytokine A1) is contained in chromosome 17 (17q12) and codes for a protein that is a member of the family of cytokines related to the subfamily of cytokines CXC, characterised by two cysteins separated by a single amino acid. This cytokine is secreted by activated T cells and shows chemotactic activity for monocytes but not for neutrophils. It binds to the receptor of chemokines CCR8.

In the context of the present invention, CCL-1 is also defined by a sequence of nucleotides or polynucleotide, which constitutes the coding sequence of the protein contained in SEQ ID NO.: 8, which would comprise several variants from:

a) nucleic acid molecules encoding a polypeptide comprising the amino acid sequence of SEQ ID NO.: 8,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 8, wherein the polypeptide encoded by said nucleic acids has the activity and structural characteristics of protein CCL-1. Among said nucleic acid molecules, the one contained in the sequence of GenBank (NCBI) NM_{—}0002981.1 or SEQ ID NO.: 7 is included.

The gene EOTAXIN-2 or chemokine (C-C motif) ligand 24 (CCL24, Ckb-6, MPIF-2, MPIF2, SCYA24, C-C motif chemokine 24; CK-beta-6; eosinophil chemotactic protein 2; myeloid progenitor inhibitor factor 2; small inducible cytokine subfamily A (Cys-Cys), member 23; small-inducible cytokine 24), that is contained in chromosome 17 (17q11.23) and codes for a protein that is a member of the family of small cytokines CC. Cytokines CC are characterised by two adjacent cysteines. The cytokine coded by this gene shows chemotactic activity against T lymphocytes, a minimum activity in neutrophils, and does not show activity for activated T lymphocytes. The protein is also a strong suppressant of the formation of colonies by multipotential hematopoietic stem cell lines.

In the context of the present invention, EOTAXIN-2 is also defined by a sequence of nucleotides or polynucleotide, which constitutes the coding sequence of the protein contained in SEQ ID NO.: 10 and would comprise several variants from:

a) nucleic acid molecules encoding a polypeptide comprising the amino acid sequence of SEQ ID NO.: 10,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 10, wherein the polypeptide encoded by said nucleic acids has the activity and structural characteristics of protein CCL-1. Among said nucleic acid molecules, the one contained in the sequence of GenBank (NCBI) NM_{—}002991.2 or SEQ ID NO.: 9 is included.

The gene PDGFAA or platelet-derived growth factor alpha polypeptide (PDGF-A, PDGF1, DGF A-chain; PDGF subunit A; PDGF-1; platelet-derived growth factor A chain; platelet-derived growth factor alpha chain; platelet-derived growth factor alpha isoform 2 preproprotein; platelet-derived growth factor subunit A) is in chromosome 7 (7p22) and codes for a protein that is a member of the family of platelet-derived growth factors. The four members of this family are mitogenic factors for cells of mesenchymal origin and are characterised by a motif of eight cysteines. The product of the gene can exist both as homodimmer and as heterodimer with the beta-polypeptide of the platelet-derived growth factor, where the dimmers are connected by disulphur bonds. Two splicing variants have been identified for this gene.

In the context of the present invention, PDGFA is also defined by a nucleotide or polynucleotide sequence, which constitutes the coding sequence of the protein contained in SEQ ID NO.: 12, which would comprise several variants from:

a) nucleic acid molecules encoding a polypeptide comprising the amino acid sequence of SEQ ID NO.: 12,
b) nucleic acid molecules, the complementary chain of which hybridises with the polynucleotide sequence of a),
c) nucleic acid molecules, the sequence of which differs from a) and/or b due to the degeneration of the genetic code,
d) nucleic acid molecules encoding a polypeptide comprising the amino acid sequence with an identity of at least 80%, 90%, or 95%, 98% or 99%, with a SEQ ID NO.: 12, wherein the polypeptide encoded by said nucleic acids has the activity and structural characteristics of protein PDGFA. Among said nucleic acid molecules, the one contained in the sequence of GenBank (NCBI) NM_{—}002607.5 or SEQ ID NO.: 11 is included.

The gene TNFRI or tumour necrosis factor receptor superfamily, member 1A (TNFRSF1A, CD120a, FPF, MGC19588, TBP1, TNF-R, TNF-R-I, TNF-R55, TNFAR, TNFR1, TNFR55, TNFR60, p55, p55-R, p60, TNF-R1; TNF-RI; TNFR-I; tumour necrosis factor binding protein 1; tumour necrosis factor receptor 1A isoform beta; tumour necrosis factor receptor superfamily member 1A; tumour necrosis factor receptor type 1; tumour necrosis factor-alpha receptor) is contained in chromosome 12 (12p13.2) and codes for a protein that is a member of the superfamily of platelet-derived growth factors. The four members of the TNF receptor, is one of the main receptors for the alpha tumour necrosis factor. This receptor can activate NF-kappaB, mediate apoptosis and work as an inflammation regulator. The antiapoptotic protein BAG4/SODD and proteins TRADD and TRAF2 have been seen to interact with this receptor and, therefore, play a role in the transduction of the receptor-mediated signal.

In this the context of the present invention, TNFIR is also defined by a sequence of nucleotides or polynucleotide, which constitutes the sequence coding the protein contained in SEQ ID NO.:14, which would comprise several variants from:

a) nucleic acid molecules encoding a polypeptide comprising the amino acid sequence of SEQ ID NO.: 14,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 14, wherein the polypeptide encoded by said nucleic acids has the activity and structural characteristics of protein TNFRI. Among said nucleic acid molecules, the one contained in the sequence of GenBank (NCBI) NM_{—}001065.3 or SEQ ID NO.: 13 is included.

The gene TNFRII or tumour necrosis factor receptor superfamily, member 1B (TNFRSF1B, CD120b, TBPII, TNF-R-II, TNF-R75, TNFBR, TNFR1B, TNFR2, TNFR80, p75, p75TNFR, TNF-R2; TNF-RII; p75 TNF receptor; p80 TNF-alpha receptor; soluble TNFR1B variant 1; tumour necrosis factor beta receptor; tumour necrosis factor binding protein 2; tumour necrosis factor receptor 2; tumour necrosis factor receptor superfamily member 1B; tumour necrosis factor receptor type II) is contained in chromosome 1 (1p36.22) and codes for a protein that is a member of the superfamily of TNF receptors. This protein and the TNF-receptor 1 form a heterocomplex that mediates the recruitment of two apoptotic proteins, c-IAP1 and c-IAP2, that have E3 ubiquitin ligase activity.

In this the context of the present invention, TNFRII is also defined by a sequence of nucleotides or polynucleotide, which constitutes the coding sequence of the protein contained in SEQ ID NO. 16 and would comprise several variants from:

a) nucleic acid molecules encoding a polypeptide comprising the amino acid sequence of SEQ ID NO.: 16,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 16, wherein the polypeptide encoded by said nucleic acids has the activity and structural characteristics of protein TNFRII. Among said nucleic acid molecules, the one contained in the sequence of GenBank (NCBI) NM_{—}001066.2 or SEQ ID NO.: 15 is included.

The gene EGF or epidermal growth factor (HOMG4, URG, beta-urogastrone; pro-epidermal growth factor) is in chromosome 4 (4q25) codes for a protein that is member of the superfamily of the epidermal growth factors.

In this the context of the present invention, EGF is also defined by a sequence of nucleotides or polynucleotide, which constitutes the coding sequence of the protein contained in SEQ ID NO.: 18 and would comprise several variants from:

a) nucleic acid molecules encoding a polypeptide comprising the amino acid sequence of SEQ ID NO.: 18,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 18, wherein the polypeptide encoded by said nucleic acids has the activity and structural characteristics of protein EGF. Among said nucleic acid molecules, the one contained in the SEQ ID NO.: 17 is included.

The gene MIP-1B or chemokine (C-C motif) ligand 4 (CCL4, CT2, AT744.1, G-26, HC21, LAG-1, LAG1, MGC104418, MGC126025, MGC126026, MIP-1-beta, MIP1B, MIP1B1, SCYA2, CYA4, C-C motif chemokine 4; CC chemokine ligand 4; G-26 T-lymphocyte-secreted protein; MIP-1-beta(1-69); PT 744; SIS-gamma; T-cell activation protein 2; lymphocyte activation gene 1 protein; lymphocyte-activation gene 1; macrophage inflammatory protein 1-beta, secreted protein G-26; small inducible cytokine A4 (homologous to mouse Mip-1b); small-inducible cytokine A4), is in chromosome 17 (17q12).

In this the context of the present invention, MIP-1B is also defined by a sequence of nucleotides or polynucleotide, forming the coding sequence of the protein contained in SEQ ID NO.: 20, which would comprise several variants from:

a) nucleic acid molecules encoding a polypeptide comprising the amino acid sequence of SEQ ID NO.: 20,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 20, wherein the polypeptide encoded by said nucleic acids has the activity and structural characteristics of protein MIP-1B. Among said nucleic acid molecules, the one contained in the sequence of GenBank (NCBI) NM_{—}002984.2 or SEQ ID NO.: 19 is included. The gene MIG or chemokine (C-X-C motif) ligand 9 (CXCL9, CMK, Humig, MIG, SCYB9, crg-10, C-X-C motif chemokine 9; gamma-interferon-induced monokine; monokine induced by gamma interferon; monokine induced by interferon-gamma; small-inducible cytokine B9) is contained in chromosome 4 (4q21).

In this the context of the present invention, MIG is also defined by a sequence of nucleotides or polynucleotide, which constitutes the coding sequence of the protein contained in SEQ ID NO.: 22, which would comprise several variants from:

a) nucleic acid molecules encoding a polypeptide comprising the amino acid sequence of SEQ ID NO.: 22,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 22, wherein the polypeptide encoded by said nucleic acids has the activity and structural characteristics of protein MIG. Among said nucleic acid molecules, the one contained in the sequence of GenBank (NCBI) NM_{—}002416.1 or SEQ ID NO.: 21 is included.

The gene MCP-1 or chemokine (C-C motif) ligand 2 (CCL2, GDCF-2, HC11, HSMCR30, MCAF, MCP-1, MCP1, MGC9434, SCYA2, SMC-CF, C-C motif chemokine 2; monocytes chemoattractant protein 1; monocytes chemoattractant protein-1; monocyte chemotactic and activating factor; monocyte chemotactic protein 1; monocyte secretory protein JE; small inducible cytokine A2 (monocytes chemotactic protein 1, homologous to mouse Sig-je); small inducible cytokine subfamily A (Cys-CYs), member 2; small-inducible cytokine A2) is in chromosome 17 (17q11.2-q12).

In this the context of the present invention, MCP-1 is also defined by a sequence of nucleotides or polynucleotide which constitutes the coding sequence of the protein contained in SEQ ID NO.: 24, which would comprise several variants from:

a) nucleic acid molecules encoding a polypeptide comprising the amino acid sequence of SEQ ID NO.: 24,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 24, wherein the polypeptide encoded by said nucleic acids has the activity and structural characteristics of protein MCP-1. Among said nucleic acid molecules, the one contained in the sequence of GenBank (NCBI) NM_{—}002982.3 or SEQ ID NO.: 23 is included.

The gene IGFBP2 or insulin-like growth factor binding protein 2 (IBP2, IGF-BP53, IBP-2; IGF-binding protein 2; IGFP-2; insulin-like growth factor-binding protein 2), is contained in chromosome 2 (2q33-q34).

In this the context of the present invention, MCP-1 is also defined by a sequence of nucleotides or polynucleotide which constitutes the coding sequence of the protein contained in SEQ ID NO.: 26, that would comprise several variants from:

a) nucleic acid molecules encoding a polypeptide comprising the amino acid sequence of SEQ ID NO.: 26,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 26, wherein the polypeptide encoded by said nucleic acids has the activity and structural characteristics of protein IGFBP2. Among said nucleic acid molecules, the one contained in the sequence of GenBank (NCBI) NM_{—}000597.2 or SEQ ID NO.: 25 is included.

The gene IGFBP1 or insulin-like growth factor binding protein 1 (IGFBP1, AFBP, IBP1, IGF-BP25, PP12, hIGFBP-1, BP-1; IGF-binding protein 1; IGFBP-1; alpha-pregnancy-associated endometrial globulin; amniotic fluid binding protein; binding protein-25; binding protein-26; binding protein-28; growth hormone independent-binding protein; insulin-like growth factor-binding protein 1; placental protein 12) is contained in chromosome 7 (7p13-p12).

In this the context of the present invention, IGFBP1 is also defined by a sequence of nucleotides or polynucleotide which constitutes the coding sequence of the protein contained in SEQ ID NO.: 28, which would comprise several variants from:

a) nucleic acid molecules encoding a polypeptide comprising the amino acid sequence of SEQ ID NO.: 28,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 28, wherein the polypeptide encoded by said nucleic acids has the activity and structural characteristics of protein IGFBP1. Among said nucleic acid molecules, the one contained in the sequence of GenBank (NCBI) NM_{—}00.596.2 or SEQ ID NO.: 27 is included.

The gene GDF-15 or growth differentiation factor 15 (GDF-15, MIC-1, MIC1, NAG-1, PDF, PLAB-PTGFB, NRG-1; NSAID (nonsteroidal anti-inflammatory drug)-activated protein 1; NSAID-activated gene 1 protein; NSAID-regulated gene 1 protein; PTFG-beta; growth/differentiation factor 15; macrophage inhibitory cytokine 1; placental TGF-beta; placental bone morphogenetic protein; prostate differentiation factor) is in chromosome 19 (19p13.11).

In this the context of the present invention, GDF-15 is also defined by a sequence of nucleotides or polynucleotide which constitutes the coding sequence of the protein contained in SEQ ID NO.: 30, which would comprise several variants from:

a) nucleic acid molecules encoding a polypeptide comprising the amino acid sequence of SEQ ID NO.: 30,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 30, wherein the polypeptide encoded by said nucleic acids has the activity and structural characteristics of protein GDF-15. Among said nucleic acid molecules, the one contained in the sequence of GenBank (NCBI) NM_{—}0048642.3 or SEQ ID NO.: 29 is included. The gene VEGFA or vascular endothelial growth factor A (RP1-261G23.1, MGC70609, MVCD1, VEGF, VPF, vascular permeability factor) is in chromosome 6 (6p12).

In this the context of the present invention, VEGF is also defined by a sequence of nucleotides or polynucleotide which constitutes the coding sequence of the protein contained in SEQ ID NO.: 32, which would comprise several variants from:

a) nucleic acid molecules encoding a polypeptide comprising the amino acid sequence of SEQ ID NO.: 32,
b) nucleic acid molecules, the complementary chain of which hybridises with the polynucleotide sequence of a),
c) nucleic acid molecules, the sequence of which differs from a) and/or b) due to the degeneration of the genetic code,
d) nucleic acid molecules encoding a polypeptide comprising the amino acid sequence with an identity of at least 80%, 90%, or 95%, 98% or 99%, with a SEQ ID NO.: 32, wherein the polypeptide encoded by said nucleic acids has the activity and structural characteristics of protein VEGF. Among said nucleic acid molecules, the one contained in the sequence of GenBank (NCBI) NM_{—}001025366.2 or SEQ ID NO.: 31 is included.

In another preferred embodiment, the detection of the quantity of any of IL-6sR, IL-1a, IL-11, CCL-1, EOTAXIN-2, PDGFAA, TNFRI, TNFRI, EGF, MIP-1B, MIG, MCP-1, IGFBP2, IGFBP1, GDF-15 and VEGF is performed by an immunoassay. The term “immunoassay”, as used in this description, refers to any analytical technique based on the reaction of the conjugation of an antibody with an antigen. Examples of immunoassays known in the state of the art are for instance, but not limited to: immnoblot, enzyme-linked immunosorbent assay (ELISA), linear immunoassay (LIA), radioimmunoassay (RIA), immunofluorescence, x-map or protein chips.

In another preferred embodiment, the immunoassay is an enzyme-linked immunosorbent assay (ELISA). The ELISA is based on the assumption that an immunoreagent (antigen or antibody) can be immobilised in a solid support, placing then this system in contact with a fluid phase that contains the complementary reagent that can be bound to a labelling compound. There are different types of ELISA: direct ELISA, indirect ELISA or sandwich ELISA:

The term “labelling compound”, as used in this description, refers to a compound that can lead to a chromogenic, fluorogenic, radioactive and/or chemoluminiscent signal, which allows the detection and quantitation of the quantity of antibodies to IL-6sR, IL-1a, IL-11, CCL-1, EOTAXIN-2, PDGFAA, TNFRI, TNFRI, EGF, MIP-1B, MIG, MCP-1, IGFBP2, IGFBP1, GDF-15 and VEGF. The labelling compound is selected from the list comprising radioisotopes, enzymes, fluorphores or any molecule susceptible of being conjugated with another molecule or detected and/or quantified directly. This labelling compound can be bound to the antibody directly or through another compound. Some examples of labelling compounds binding directly are, though not limited to, enzymes such alkaline phosphatase or peroxidase, radioactive isotopes such as ³²P or ³⁵S, fluorochromes such as fluorescein or metal particles, for direct detection by colorimetry, auto-radiography, fluorimetry or metalography, respectively.

Another aspect of the invention refers to a method of diagnosis, prognosis and classification of individuals, hereinafter referred to second method of the invention, comprising the steps (i)-(iii) according to the first method of the invention and that also comprises assigning the individual of step (i) to the group of individuals without COPD or lung cancer when there is no expression of the genes IGFBP1, MIP1β, CCL-1, MIG and PDGFAA.

In a preferred embodiment, the second method of the invention comprises the steps (i)-(iii) according to the first method of the invention and also comprises assigning the individual of step (i) to the group of individuals with adenocarcinoma, when there is an increase in the expression product of genes IL-11 and/or CCL-1, in relation to a reference quantity.

In another preferred embodiment, the second method of the invention comprises steps (i)-(iii) according to the first method of the invention and also comprises assigning the individual of step (i) to the group of individuals with COPD when expression of MIG is detected and expression of CCL-1 and IGFBP1 is not detected.

In another preferred embodiment, the second method of the invention comprises the steps (i)-(iii), according to the first method of the invention, and also comprises assigning to the individual of step (i) to the group of individuals with adenocarcinoma when detecting the expression of CCL-1 and the amount of expression of MIP1β is lower than 25 pg/ml, more preferably lower than 22 pg/ml and even more preferably lower than 20 pg/ml.

In another preferred embodiment, the second method of the invention comprises the steps (i)-(iii), according to the first method of the invention, and also comprises assigning the individual of step (i) to the group of individuals with squamous carcinoma when detecting the expression of PDGFAA or MIP1β at any level and the expression of CCL-1 is not detected.

In another preferred embodiment, the second method of the invention comprises the steps (i)-(iii), according to the first method of the invention, and also comprises assigning the individual of step (i) to the group of individuals with COPD and adenocarcinoma when detecting the expression of CCL-1 at any level and the expression of VEGF is lower than 240 pg/ml, more preferably lower than 220 pg/ml and even more preferably lower than 200 pg/ml.

In another preferred embodiment, the second method of the invention comprises the steps (i)-(iii), according to the first method of the invention, and also comprises assigning the individual of step (i) to the group of individuals with COPD and squamous adenocarcinoma when detecting the expression of GDF-15 higher than 40 pg/mL, and more preferably higher than 50 pg/mL and VEGF higher than 180 pg/ml, more preferably lower and 200 pg/ml, respectively, and the expression of CCL-1 or EGF is not detected.

There is currently no cure for COPD; however, it is a preventable, manageable disease. For the treatment of COPD, the strategies most commonly used are to quit smoking, rehabilitation and drug therapy (often the use of inhalers). Some patients will require long-term treatment with oxygen or a lung transplant.

Therefore, bronchodilators are the drugs relaxing the smooth muscle of the airways, which increases airway calibre and improves airflow, reducing the symptoms of breathlessness, etc., resulting in a better quality of life in people with COPD. However, they do not reduce the progression of the underlying disease. Bronchodilators are generally administered with an inhaler or through a nebuliser.

There are two main types of bronchodilators, β2 agonists and anticholinergics. Anticholinergics appears to be superior to β2-agonists for COPD. Anticholinergics reduce death from respiratory causes, while β2-agonists have no effect on mortality by respiratory diseases. Each type can be long-lasting (with an effect lasting 12 hours or longer) or short-acting (with a fast onset of action not lasting long).

β2-agonists

β2-agonists stimulate receptors in the bronchial smooth muscle, relaxing it. Several β2-agonists are available. Salbutamol (trade name: Ventolin) and terbutalin are widely used as short-acting β2-agonists, providing fast relief of COPD symptoms. Long-acting β2-agonists (LABA) such as salmeterol and formoterol are used as maintenance therapy and their use involves a better air flow, improves capacity for exercise and quality of life.

### Anticholinergics

Anticholinergics relax the airways by blocking stimulation of cholinergic nerves. Ipratropium provides a short action and fast relief of COPD symptoms. Tiotropium is a long-acting anticholinergic with a regular use associated with improved air flow, capacity for exercise, and quality of life. Ipratropium is associated with cardiovascular morbidity.

### Corticosteroids

Corticosteroids are used as tablets or inhaled to treat and prevent acute COPD episodes. Inhaled corticosteroids (ICS) have not been shown to be beneficial for people with mild COPD; however, they have been shown to reduce acute worsening in individuals with moderate or severe COPD. However, they are associated with higher rates of pneumonia.

### Other Medicaments

Theophylline is a bronchodilator and a phosphodiesterase inhibitor that, at high doses, can reduce the symptoms in some people with COPD. The side effects such as nausea and heart stimulation limit their use. Phosphodiesterase-4 antagonists roflumilast and cilomilast have completed the phase 2 of clinical trials. Tumour necrosis factor antagonists, such as infliximab, suppress the immune system and reduce inflammation. Infliximab has been tested in patients with COPD, but there was no evidence of benefit.

### Lung Cancer

The common treatments include surgery, chemotherapy and radiation therapy. NSCLC (non-small-cell lung carcinoma) is treated with surgery, while SCLC (small-cell lung carcinoma) generally responds better to chemotherapy and radiation therapy. This is in part because SCLC is often disseminated very early, and these treatments are better when reaching the cancer cells disseminating to other parts of the body.

### Treatment of Lung Cancer

The treatment for lung cancer depends on the type of cancer, its dissemination and the patient's condition. Common treatments include palliative care, surgery, chemotherapy and radiation therapy.

### Surgery

If the investigations confirm non-small cell lung cancer, the scenario must be re-evaluated to establish whether the disease is localised and is amenable of surgery or has been disseminated to the point that it cannot be cured with surgery. For this, computerised tomography and positron emission tomography (PET) are used. Blood tests and pulmonary function tests are also necessary to evaluate if the patient is well enough to be operated. If the pulmonary function tests show a defective respiratory reservoir, surgery may be contraindicated.

In most cases of the first stages of non-small cell lung cancer, the removal of a lung lobe (lobectomy) is the surgical treatment of choice. In patients not eligible for total lobectomy, a small sublobar removal (wedge removal) can be performed. However, wedge resection shows a higher risk of relapse of the disease than lobectomy. Brachytherapy with radioactive iodine in the wedge removal margins can reduce the risk of relapse. Rarely removal of a whole lung is performed (pneumonectomy).

Guided thoracoscopic video surgery and video-guided lobectomy use a minimally invasive approach of the surgery of lung cancer. VATS lobectomy is also effective as compared to conventional open lobectomy and with a shorter post-operative period of the disease.

In small-cell lung cancer (SCLC), chemotherapy and/or radiation therapy are commonly used. However, the role of surgery in SCLC is being re-evaluated. Surgery can improve the results when chemotherapy and radiation therapy are added in the early stage.

### Radiation Therapy

Radiation therapy is often administered with chemotherapy and can be used for curing purposes in patients with non-small cell lung carcinoma not eligible for surgery. This high-intensity radiation therapy method is called radical radiation therapy. An improvement of this technique is continuous accelerated hyperfractionated radiation therapy, where a high dose of radiation therapy is given in a short time period. For small-cell cases, the cases of carcinoma of the lung that can be potentially cured, chest radiation in addition to chemotherapy. Is recommended. Post-operative chest radiation therapy in generally must not be used after curative intent surgery for non-small cell lung carcinoma.

If cancer growth blocks a short section of the bronchi, brachytherapy (localised radiation therapy) can be administered directly into inside the airways to open the way. [As compared to external radiation therapy, brachytherapy allows to reduce the treatment time and reduce exposure to radiation of the health personnel.

Prophylactic cranial irradiation (PCI) is a type of radiation therapy in the brain that is used to reduce the risk of metastasis. PCI is more useful in small-cell lung cancer.

Recent advances in orientation and images have led to the development of stereotactic radiation for the treatment of lung cancer in early stages. In this form of radiation therapy, high doses are delivered in a small number of sessions by stereotaxis. It is used mainly in patients not eligible for surgery due to medical comorbidities.

Therefore, in patients with non-small and small cell lung cancer, lower doses of chest radiation can be used for controlling symptoms (palliative radiation therapy).

### Chemotherapy

The chemotherapy regimen depends on the type of tumour.

### Small-Cell Lung Carcinoma.

Although in a relatively early stage, small-cell lung cancer is treated mainly with chemotherapy and radiation therapy. In small-cell lung cancer, the chemotherapeutic agents most commonly used are cisplatin and etoposide. Their combinations with carboplatin, gemcitabine, paclitaxel, vinorelbine, topotecan, irinotecan are also used.

### Non-Small Cell Lung Cancer

In advanced non-small cell lung cancer, chemotherapy improves survival and is used as first-line therapy, provided the patient is well enough to receive the treatment. Two medicaments are generally used, one of which is often based on platinum (cisplatin or carboplatin). Other drugs used are gemcitabine, paclitaxel and docetaxel.

Advanced non-small cell lung carcinoma is often treated with cisplatin or carboplatin, in combination with gemcitabine, paclitaxel, docetaxel, etoposide or vinorelbine. Pemetrexed has been also recently used.

### Adjuvant Chemotherapy

Adjuvant chemotherapy refers to the use of chemotherapy after an apparently curative surgery to improve the outcome. In non-small cell lung carcinoma, samples are taken during surgery of the close lymph nodes. If phase II or III of the disease is confirmed, adjuvant chemotherapy improves five-year survival by 5%. The combination of vinorelbine and cisplatin is more effective than the old therapeutic regimens.

Adjuvant chemotherapy for patients with stage IB cancer is controversial, as clinical trials have not shown clearly a survival benefit. Pre-operative chemotherapy studies (neoadjuvant chemotherapy) in non-small cell lung cancer that can be operated have not been conclusive.

### Chemotherapy

In patients with lung cancer in stage 3, that cannot be removed by surgery, treatment combined with radiation therapy and chemotherapy improves survival significantly.

### Targeted Therapy

In recent years, several specific molecular therapies have been developed for the treatment of advanced lung cancer. Gefitinib (Iressa) is one of these drugs, focusing on the domain of tyrosine kinase of the epidermal growth factor receptor (EGF)R, expressed in many cases of non-small cell lung cancer. It has not been shown to increase survival, though women, Asian people, non-smokers and people with bronchialveolar carcinoma appear to obtain the maximum benefit from gefitinib.

Erlotinib (Tarceva), another EGFR tyrosine kinase inhibitor, increases survival in non-small cell lung cancer, and was approved by the FDA in 2004, as second-line treatment for it. As with gefitinib, it also appears to work better in women, Asian patients, non-smokers and people with bronchiolalveolar carcinoma, in particular those with specific mutations in EGFR.

The angiotensin inhibitor bevacizumab (Avastin) (in combination with paclitaxel and carboplatin) improves the survival of patients with non-small cell lung carcinoma. However, this increases the risk of bleeding of the lungs, in particular in patients with squamous cell carcinoma.

Advances in cytotoxic drugs, pharmacogenetics and drug design appear to be promising. A number of targeted agents are in the early stages of clinical research, such as cyclooxygenase-2 inhibitors, the promoter of apoptosis exisulind, proteasoma inhibitors, bexarotene, the epidermal growth factor receptor inhibitor cetuximab, and vaccines. Crizotinib has been shown to be a significant promise in the first clinical trials in a sub-group of non-small cell lung carcinoma characterised by the fusion oncogene EML4-ALK that is found in some relatively young patients, slightly or never smokers, with adenocarcinoma. The future research areas include proto-oncogene as inhibitors, inhibition of phosphoinisitide-3-kinase, inhibition of histone deacetylase, and replacement of the tumour suppressant gene.

Therefore, another aspect of the invention refers to the use of a pharmaceutical composition comprising an active ingredient selected from β2-agonist, an anticholinergic, a compound from the group of corticosteroids, a phosphodiesterase inhibitor and an immune system suppressor, in the preparation of a medicament for the treatment of an individual with COPD that can be identified by the method of the invention.

Another aspect of the invention refers to the use of a pharmaceutical composition that comprises an active ingredient selected from coordination complexes of platinum (cisplatin or carboplatin), gemcitabine, paclitaxel, docetaxel, etoposide, vinorelbine, pemetrexed, gefitinib, erlotinib, bevacizumab or any of its combinations in the manufacture of a medicament for the treatment of an individual with adenocarcinoma and/or squamous carcinoma, associated or not with COPD, that can be identified by the method of the invention.

As used herein, the term “active ingredient”, “active substance”, “pharmaceutically active substance”, “active ingredient” or “pharmaceutically active ingredient” means any component that potentially provides a pharmacological activity or any different effect in the diagnosis, cure, mitigation, treatment or prevention of a disease, or that affects the structure or function of the body of humans or other animals. The term includes components promoting a chemical change in the production of the drug and are present in it in a planned modified form providing the specific activity or effect.

Another aspect of this invention refers to a kit or device, hereinafter referred to as the kit of the invention, comprising the elements necessary to quantify the expression of cytokines and growth factors selected from IL-6sR, IL-1a, IL-11, CCL-1, EOTAXIN-2, PDGFAA, TNFRI, TNFRII, EGF, MIP-1B, MIG, MCP-1, IGEFBP2, IGFBP1, GDF-15, VEGF or any combinations thereof.

In a preferred embodiment, the kit comprises the elements necessary to quantify the expression of IGFBP1, MPI1β, CCL-1, MIG, PDGFAA, GDF-15, VEGF and EGF.

In a preferred embodiment, the kit comprises the elements necessary to quantify the expression of cytokines and growth factors CCL-1 and/or IL-11. More preferably, the kit or device comprises the antibodies anti-CCL-1 and anti-IL-11.

Even more preferably, the kit of this invention comprises antibodies that are selected from the listing consisting of: antibodies anti-IL-6sR, anti-IL-1a, anti-IL-11, anti-CCL-1, anti-EOTAXIN-2, anti-PDGFAA, anti-TNFRI, anti-TNFRII, anti-EGF, anti-MIP-1B, anti-MIG, anti-MCP-1, anti-IGFBP2, anti-IGFBP1, anti-GDF-15, anti-VEGF or any of the combinations thereof. More preferably, the kit comprises the antibodies anti-IGFBP2, anti-MIP-1B, anti-CCL-1, anti-MIG, anti-PDGFAA, anti-GDF-15, anti-VEGF or anti-EGF.

In another preferred embodiment, the kit of the invention comprises secondary antibodies or positive and/or negative controls. The kit can also include, with no other type of limitation, buffers, solutions of protein extraction, agents to prevent contamination, protein degradation inhibitors, etc.

On the other hand, the kit can include all supports and containers necessary for set-up and optimisation. The kit preferably comprises also the instructions to perform the methods of the invention.

Another aspect refers to the use of the kit of the invention, for the diagnosis, prognosis and classification of the a) individuals with no COPD or lung cancer, b) individuals with COPD, c) individuals with adenocarcinoma, d) individuals with squamous carcinoma, e) individuals with COPD and adenocarcinoma, or f) individuals with COPD and squamous carcinoma.

Another aspect of the invention relates to a computer readable storage means comprising software instructions that can make that a computer performs the steps of any of the methods of the invention (of the first or second method of the invention).

In a preferred embodiment, the computer readable storage means comprise at least one of the antibodies anti-IL-6sR, anti-IL-1a, anti-IL-11, anti-CCL-1, anti-EOTAXIN-2, anti-PDGFAA, anti-TNFRI, anti-TNFRII, anti-EGF, anti-MIP-1B, anti-MIG, anti-MCP-1, anti-IGFBP2, anti-IGFBP1, anti-GDF-15 and anti-VEGF or any of its combinations. In another more preferred embodiment, the computer readable storage means comprises the antibodies anti-IGFBP1, anti-MIP1β, anti-CCL-1, anti-MIG, anti-PDGAA, anti-GDF-15, anti-VEGF and anti-EGF.

The methods of the invention can include additional steps, for instance, the separation of proteins by mono and bidimensional electrophoresis (2D-PAGE) or previous digestion with trypsin of a mixture of proteins (of the sample) for then purifying and analysing the peptides by mass spectrometry (MS), such as MALDI-TOF or by multidimensional chromatography, by ICAT (Isotope-coded affinity tags), DIGE (Differential gel electrophoresis) or protein arrays.

In another preferred embodiment, the computer readable storage means comprises oligonucleotide or single-channel microarrays designed from a known sequence or an mRNA of at least one of the genes IL-6sR, IL-1a, IL-11, CCL-1, EOTAXIN-2, PDGFAA, TNFRI, TNFRII, EGF, MIP-1B, MIG, MCP-1, IGFBP2, IGFBP1, GDF-15 and VEGF or any of the combinations thereof. Even more preferably the computer readable storage means comprises oligonucleotides or single-channel microarrays designed from a known sequence or an mRNA of the genes IGFBP1, MIP1β, CCL-1, MIG-PDGFAA, VEGF and EGF.

For instance, the oligonucleotide sequences are built in the surface of the chip by the sequential elongation of a growing chain with a single nucleotide using photolithography. Therefore, oligonucleotides are anchored by edge 3′ using a selective nucleotide activation method, protected by a photolabile reagent, by selective light incidence through a photomask. The photomask can be physical or virtual.

Therefore, oligonucleotide probes can be from 10 to 100 nucleotides, more preferably, from 20 to 70 nucleotides, and even more preferably, from 24 to 30 nucleotides. For the quantitation of the gene expression, about 40 oligonucleotides per gene are used approximately.

Synthesis in situ on a solid support (for instance, glass) could be made by ink-jet technology, which requires longer probes. The supports could be, but without limitation, NC or nylon (charged) filters or membranes, silicon or glass-holders for microscopes covered with aminosilanes, polylysine, aldehydes or epoxy. The probe is each of the chip samples. The target is the sample to be analysed: messenger RNA, total RNA, a PCR fragment, etc.

Another aspect the invention refers to a transmissible signal comprising software instructions that can do that a computer fulfils the steps of any of the methods of the invention.

The terms “polynucleotide” and “nucleic acid” are used here exchangeably, referring to polymeric forms of nucleotides of any length, both ribunucleotides (RNA or DNA) and deoxyribonucleotides (DNA).

The terms “amino acid sequence”, “peptide”, “oligopeptide”, “polypeptide” and “protein” are used here exchangeably and refer to a polymeric form of amino acids of any length, that can be coding or not coding, chemically or biochemically modified.

During the description and the claims the word “comprises” and its variants do not intend to exclude other technical characteristics, additives, components or steps. For those skilled in the art, other objects, advantages and characteristic of the invention will be concluded in part of the description and in part of the practice of the invention. The following examples and drawings are provided by way of illustration and it is not intended that they limit this invention.

### BRIEF DESCRIPTION OF THE FIGURES

FIG. 1. Hierarchical cluster of association of components.

FIG. 2. Analysis of the significant differences of expression of the 16 proteins of interest by calculation of the p value, p<0.05,* p<0.01,** p<0.001.***

FIG. 3. Analysis of the 16 proteins of interest by Western blot with specific antibodies.

FIG. 4. Validation of the differential expression of the proteins of interest by ELISA.

FIG. 5. Analysis of sensitivity and specificity of the proteins of interest, for each group of the conditions evaluated, from the expression data obtained by the ELISA methodology.

FIG. 6. Study profile.

FIG. 7. A. Grouping heat map of 80 supervised proteins differentially expressed between the control group and the disease groups. Analysis dendrograms of grouping of samples and proteins are shown in the upper and left part, respectively. The relative up and down regulation of the protein is shown in red and blue, respectively. B. Levels of expression of 16 interesting proteins after map analysis. The expression levels of measurement of each protein, over the mean level of expression through the condition. The error bars represent median values, p<0.05,* p<0.01,**, p<0.005.***

FIG. 8. The candidate proteins selected CCL-1 and IL-11 were validated by Western blot and ELISA. A. Western transfer analysis of IL-11 and proteins CCL-1 in different samples. B. Analysis of levels of protein of IL-11 and CCL-1 by ELISA in the first validation cohort. The black horizontal lines are the medians. C. Analysis of the protein levels of IL-11 and CCL-1 by ELISA in the second validation cohort. The black horizontal lines are the median.

FIG. 9. ROC curves for IL-11 and CCL-1. A. ROC curves for IL-11 and CCL-1 in patients with adenocarcinoma versus all groups of the first validation cohort B. ROC curves for IL-11 and CCL-1 I patients with adenocarcinoma versus all groups of the second validation cohort.

FIG. 10. Rate of positive results for IL-11 and CCL-1 in patients with adenocarcinoma. A. Rate of positive results for IL-11, CCL-1, IL-11 and CCL-1, IL-11 and/or CCL-1 in all patients with adenocarcinoma in the first cohort. B. Rate of positive results for IL-11, CCL-1, IL-11 and CCL-1, IL-11 and/or CCL-1 in all patients with adenocarcinoma in the additional cohort.

### EXAMPLES

### Examples of Embodiment of the Invention

The invention shall be illustrated below by tests performed by the inventors.

### Example 1

### Determination of Cytokines with Differential Expression

### Patients and Samples

A total of 141 samples from bronchoalveolar lavage (BAL) from four different patient groups (patients with COPD, with LC, with COPD and LC and without COPD or LC) of the year 2009 to 2011 were analysed.

The samples were divided into two groups. The first sample group of 60 patients was used to perform the study. A description of all patients include are given in Table 1. The second group, of 81 patients, was used for validation of the results (Table 2). All samples were collected at the Hospital Virgen del Rocío (Seville, Spain) from patients requiring flexible bronchoscopy for diagnostic purposes. This study was approved by the Ethics Committee of the Hospital and a written informed consent was obtained from all patients before inclusion in the study.

The subjects were prepared with a combination of topical anaesthesia (20% benzocaine aerosol into the pharynx plus 2% topical lidocaine as required) and conscious sedation with midazolam and meperidine according to institutional guidelines. The bronchoalveolar lavage (BAL) samples were obtained from instillation and aspiration of 40 to 60 mL 0.9% aliquots of sterile saline solution in the bronchopulmonary segment. The fluid recovered was immediately run through a 100 micra filter of sterile nylon (Beton Dickinson, San Jose, Calif.) to clear the mucus, subsequently transported in ice to the laboratory. The total volume was centrifuged for 10 minutes at 1800×g and 4° C. The supernatant was divided into aliquots in 2 mL tubes and frozen at −80° C. until subsequent use.

### Handling the Sample

The tests were performed in about 4.8 mL of the sample. Due to their low protein content, the BAL samples were concentrated before use, using a vacuum concentrator (Concentrator plus—Eppendorf, Hamburg, Germany). For this, they were unfrozen in ice adding a cocktail of protease inhibitors (Thermo Scientific, Franklin, Mass., US). The initial volume of the samples was reduced to 250-450 μL in 2.6 hours. Protein quantitation was established by the RCDC method (Bio-Rad, Hercules, Calif., US).

### Protein Arrays

For the purpose of studying the protein profiles of the four groups of patients, an antibody array, a series kit available in the market that analyses the expression levels of 80 cytokines and growth factors (Quantibody® matrix of human antibodies of cytokines 1000—RayBioetech, Norcross, Ga., US) were used. The analysis of antibodies was performed according to the instructions provided by manufacturer. In short, the microarray matrices were incubated with the block buffer at room temperature for 30 minutes and then with the sample for 120 minutes. The microarray matrices were washed with wash solution I (Wash Buffer I) three times and with the wash II buffer II for 2 hours at room temperature (5 minutes per wash). Then the microarray matrices were incubated with the cocktail of antibodies at room temperature for 120 minutes. Finally, the microarray matrices were washed and incubated with Cy3 equivalent to conjugated streptavidine at room temperature for 120 minutes. The intensity measurements equivalent to the expression of each protein were viewed through a laser scanner GenePix 4100 A (Molecular Devices, Sunnyvale, Calif., US). The intensities of each protein were measured four times in each copy.

### Western Blot

Fifty μg of proteins from BAL were separated into gels between 7 and 12%, depending on the protein size, by electrophoresis in polyacrylamide gels with sodium sulphate dodecyl (SDS-PAGE) and subsequently transferred to polyvinylidene fluoride (PVDF) membranes (Bio-Rad, Hercules, Calif., US), blocked with a 5% BSA solution and incubated all night long at 4° C. with primary antibodies according to the instructions of the manufacturer: anti-EGF (1:500, Santa Cruz, Calif., US), anti VEGF, anti-IGFPB1, anti-IGFBP2, anti-eotaxin2 (CCL24), anti-MIP-1β (CCI4), anti-MIG, anti-GDF15 (CXCL9), anti-PDGFAA, anti-IL6R, anti-IL1R1, anti-IL11 and anti-1309 (1:200 Abcam, Cambridge, Mass., US), anti-TNFRI, anti-TNFRII and anti-MCP-1 (1:1000 Cell signalling, Beverly, Mass., US). Consecutively, they were incubated with secondary antibodies, conjugated with anti-mouse (GE Healthcare, Uppsala, Sweden) and anti-rabbit peroxidase (Cell signalling, Beverly, Mass., US), applied to the individual membranes (1:2000) for one hour at room temperature. The bands corresponding to the proteins of interest were revealed by hemoimmunoluminiscence using ECL (GE Healthcare, Uppsala, Sweden) and were viewed in image analyser (Mini LAS-3000, Fujifilm, Tokyo, Japan). The relative protein levels were calculated as compared to the quantity of protein β-actin (1:1000 Abcam, Cambridge, Mass., US). The tests were repeated three times independently.

### ELISA

The ELISA tests were performed using specific antibodies in BALF samples of the second cohort of patients (validation cohort), sandwich ELISA tests were used for CCL4/MP-1β, CXCL9/MIG, IGFBP-2, IGFBP-1, EGF, RI/TNFRSF1A sTNF, RI/TNFRSF1A sTNF, IL-1, RI, IL-6 Rα, CCL2/MCP-1, VEGF, GDF-15, CCL24/Eotaxin-2/MPIF-2, CCL1/I-309, IL-11, PDGF-AA (DuoSet, R & D Systems, Minneapolis, Minn., US). All the samples were analysed in duplicate. The limits of detection for these tests were established form 25 to 200 μg of proteins.

The protocol for performing ELISA was as follows. The capture antibody was first diluted in PBS and 100 μL of it were added to each well of a 96-well plate, which was incubated all night long at 4° C. Subsequently it was washed four times in TBS with 0.05% of Tween-20 (0.05% TBST). Then, it was blocked with 1% bovine serum albumin for one hour at room temperature, again washed four times with 0.05% of TBST. Consecutively, the samples and standards of protein were added, and it was incubated for 2 hours at room temperature. After these steps the detection antibody was added diluted in PBS, for 2 hours at room temperature. Finally, streptavidine (DuoSet, R&D Systems) was added and the plate was incubated for 30 minutes. Finally, the colouring reagent o-phenylenediamine was added and the reaction was performed in darkness for 20 minutes. After this time 2N H2SO4 was added to each well to stop the reaction and absorbance was read in a plate reader at 450 nm (Emax, Molecular Devices, Minneapolis, Minn., US). The absorbance measurements were extrapolated to a standard line obtained with the protein standards to establish the protein concentration in the sample measured.

### Results Obtained

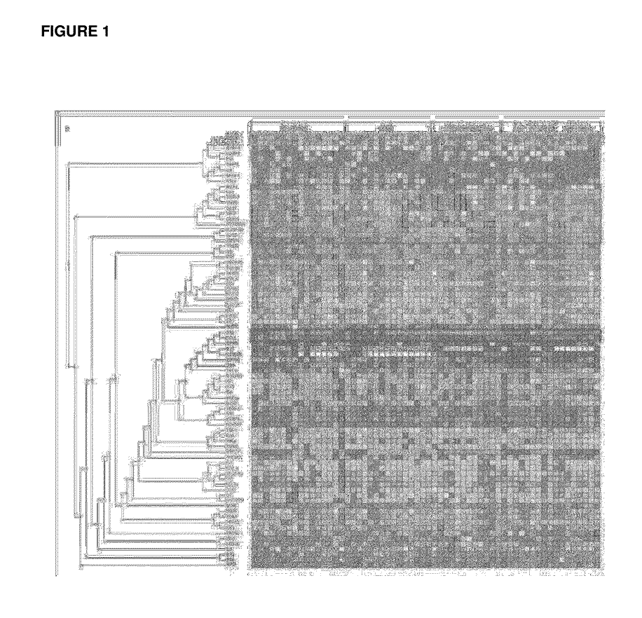

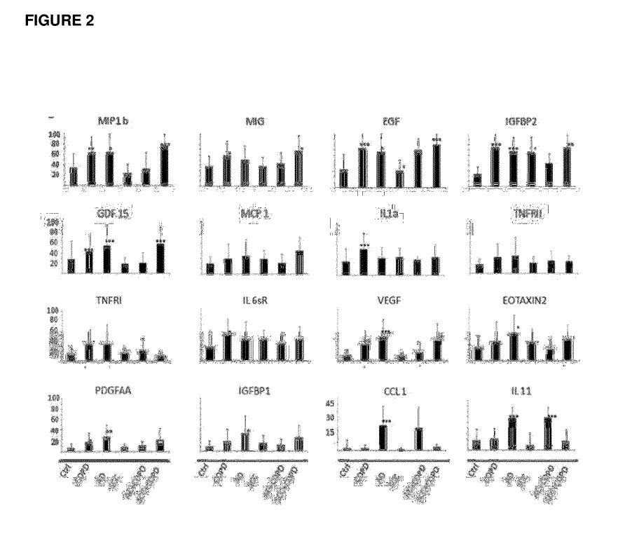

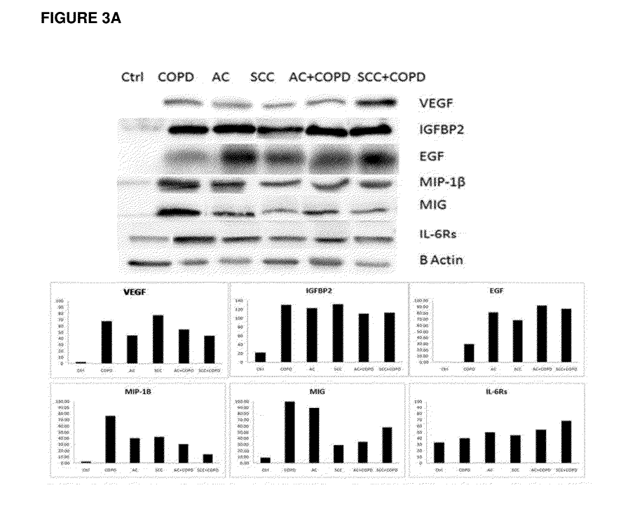

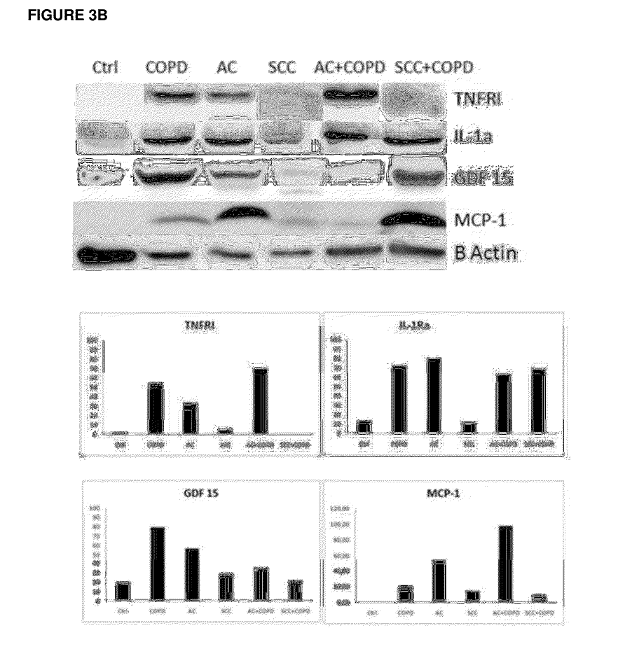

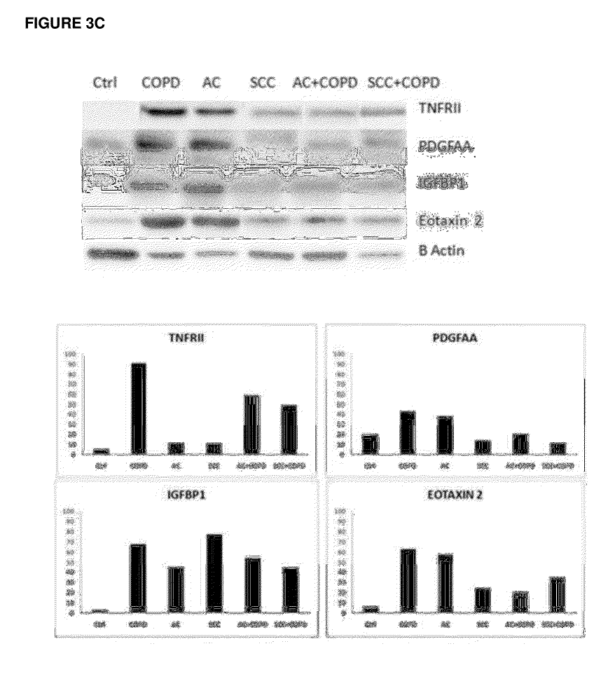

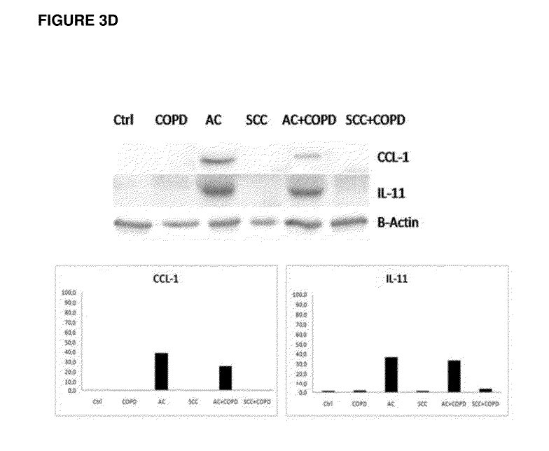

The first step to perform the study was to perform arrays of antibodies (80 cytokines and growth factors involved in the inflammatory response) in the 60 patients collected in Table 1. The data obtained from the reading of arrays were analysed by building a supervised hierarchical cluster using the Euclidean distance as method of association of components and by the use of the statistical analysis software babelomics version 4.2 (Mínguez P and J Dopazo) (FIG. 1). The expression level of each protein, in relation to the mean expression level in all conditions, was represented in values of 0-100%. From this analysis we highlight 16 proteins of interest (CCL4/MIP-1β, CXCL9/MIG-IGFBP-2, IGFBP-1, EGF, RI/TNFRSF1A sTNF, RI/TNFRSF1A sTNF, IL-1 RII, IL-6 Rα, CCL2/MCP-1, VEGF, GDF-15, CCL24/Eotaxin-2/MPIF-2, PDGF-AA CCL1/I-309, IL-11), which show expression differences in the different disease groups tested versus the control group (non-COPD, non-LC). For instance, proteins CCL1 and IL-11 are only expressed in adenocarcinoma or adenocarcinoma with COPD.

In parallel, the mean and standard deviation were analysed for each protein of interest for each condition studied and for the control group, wherein p<0.05,*, p<0.01,** p<0.001*** (FIG. 1). Observing significant differences similar to those existing between the different pathological groups versus the control group in the analysis of cluster. This suggests that this selection of proteins could help us discriminate between some types of diseases and others, as each of these proteins has a different expression from the control group depending on the type of disease.

Then it was intended to continue to evaluate further the relationship between the proteins selected and their possible involvement in the diseases studied. It was proposed to validate the findings by using another method different from the previous; for this, we used Western blot methods. By this technique the expression of each protein selected in BAL samples from patients selected in Table 1 (FIG. 1) was measured. The results obtained are consistent with those previously obtained, so that it appears to be increasingly evident that the proteins selected are involved to a higher or lower degree in the disease studied herein.

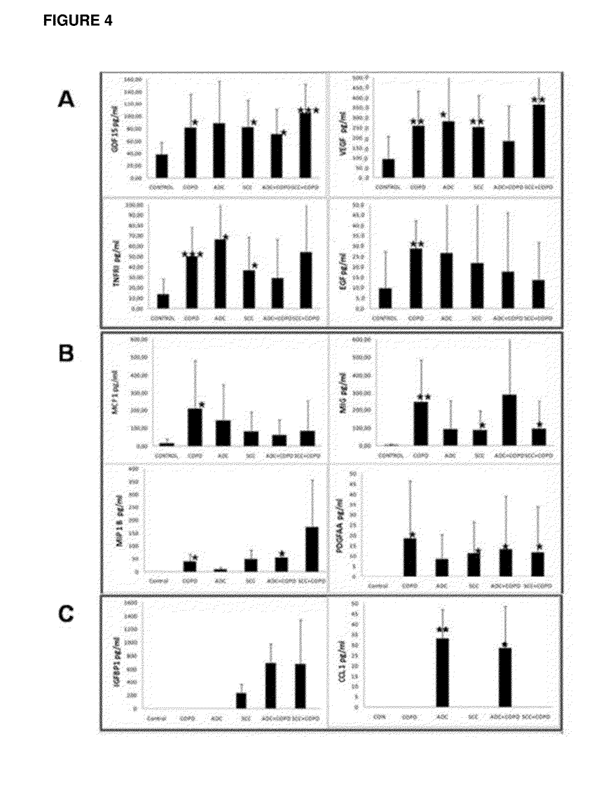

The next step was to obtain a sufficiently robust profile, susceptible of being used in the daily clinical practice. To fulfil this objective, a new patient cohort was used, with characteristics similar to those of the previous cohort, with the only change of an increase in the number of patients of the group of LC and LC/COPD. This increase is due to the need for clarifying the differences previously seen in the previous studies, between the two types of LC (ADE and SCC). For measuring proteins, the ELISA method was used, as its use is widely spread in the daily clinical practice (FIG. 4). Thanks to this analysis it was seen, on the one hand, that not all proteins are involved in all pathological groups as shown in the previous analyses, and that there is a group of four proteins (GDF15, VEGF, EGF, TNFR I) that show significant expression differences from the control group, other 4 proteins (MIG, MIP-1B, MCP1, PDGFAA) that virtually are not expressed control situation. However, they show significant differences in the various pathological groups, and two proteins (IGFBP1, CCL1) that are only expressed in some pathological groups and, on the other hand, this analysis helps establish a more robust protein profile than the previous, if possible, susceptible of being extrapolated as a kit to the clinical practice (FIG. 4).

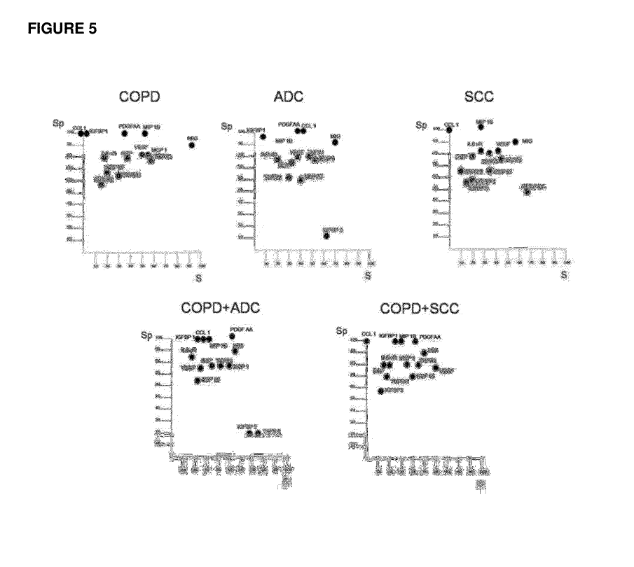

Subsequently the potential of each protein was analysed as possible biomarkers in each condition tested. For this, sensitivity, specificity, positive predictive value and negative predictive value were studied from the results obtained in the above section (FIG. 5). This analysis evidences that the MIG protein could be used as biomarker, MIG shows a sensitivity and specificity around 90% in the BAL samples of patients with COPD, while proteins CCL1 and IGFBP1 show a sensitivity of 0% and a specificity of 100%, which would indicate that these three proteins could diagnose this condition by conventional ELISA. In the same way, protein CCL1 shows a specificity of 100% and a sensitivity of 40%, so it could be a discriminating biomarker for samples from adenocarcinoma. This analysis consolidates the protein profile that we have obtained through the different analyses, helping us establish a possible kit that can discriminate the patients in the different study groups.

Finally, a logistic regression model was used by calculation of the odds ratio, to define the possible diagnostic kit. This allowed to associate if the presence or absence of some proteins was related to the probability of having a given aetiology (Table 4).

TABLE 1
Characteristics of the first patient cohort

| | Control | | COPD | | LC | | COPD with LC | |
|---|---|---|---|---|---|---|---|---|
| | n = 16 | | n = 15 | | n = 17 | | n = 12 | |
| Gender | | | | | | | | |
| Male | 100.0% | (16) | 100.0% | (15) | 100.0% | (17) | 100.0% | (12) |
| Female | 0.0% | (0) | 0.0% | (0) | 0.0% | (0) | 0.0% | (0) |
| Mean age | 61.3 | [41-80] | 61.5 | [45-78] | 60.7 | [46-69] | 60.7 | [49-68] |
| [range] | | | | | | | | |
| Status | | | | | | | | |
| Smoker | 68.8 | (11) | 53.3% | (8) | 52.9% | (9) | 83.3% | (10) |
| Former-smoker | 31.2% | (5) | 46.7% | (7) | 47.1% | (8) | 16.7% | (2) |

| Packs-year | 21.82 | 32.2 | | 35.21 | 30.78 | |
|---|---|---|---|---|---|---|
| COPD | | | | | | |
| 1 | — | 20.0% | (3) | — | 58.3% | (7) |
| 2 | — | 33.3% | (5) | — | 25.0% | (3) |
| 3 | — | 26.7% | (4) | — | 0.0% | (0) |
| 4 | — | 20.0% | (3) | — | 16.7% | (2) |

| Lung cancer | | | | | | |
|---|---|---|---|---|---|---|
| ADC | — | — | 70.6% | (12) | 33.3% | (14) |
| SCC | — | — | 29.4% | (5) | 66.7% | (8) |

TABLE 2
Characteristics of the second patient cohrt

| | Control | | COPD | | LC | | COPD with LC | |
|---|---|---|---|---|---|---|---|---|
| | n = 10 | | n = 13 | | n = 24 | | n = 34 | |
| Gender | | | | | | | | |
| Male | 60.0% | (6) | 84.6% | (11) | 83.3% | (20) | 95.5% | (32) |
| Female | 40.0% | (4) | 15.4% | (2) | 16.7% | (4) | 4.5% | (0) |
| Mean age | 52.3 | [42-58] | 65.2 | [51-75] | 61.2 | [48-75] | 64.3 | [48-75] |
| [range] | | | | | | | | |
| Status | | | | | | | | |
| Smoker | 100.0% | (10) | 53.8% | (7) | 50.0% | (12) | 53.0% | (18) |
| Former-smoker | 0.0% | (0) | 46.2% | (6) | 50.0% | (12) | 47.0% | (16) |

| Packs-year | 21.82 | 34.2 | | 33.21 | 39.78 | |
|---|---|---|---|---|---|---|
| COPD | | | | | | |
| 1 | — | 30.8% | (4) | — | 26.5% | (9) |
| 2 | — | 53.8% | (7) | — | 50.0% | (27) |
| 3 | — | 15.4% | (2) | — | 23.5% | (8) |
| 4 | — | 0.0% | (0) | — | 0.0% | (0) |

| Lung cancer | | | | | | |
|---|---|---|---|---|---|---|
| ADC | — | — | 37.5% | (9) | 47.0% | (16) |
| SCC | — | — | 62.5% | (15) | 53.0% | (18) |

TABLE 3
Quantity of proteins used in ELISA

| PROTEIN | μg |
|---|---|
| GDF-15 | 25 |
| TNFRI | |
| VEGF | |
| EGF | |
| MIG | 50 |
| MCP-1 | |
| IGFBP-1 | |
| PDGFAA | 75 |
| MIP1β | 125 |
| CCL-1 | 200 |

TABLE 4
Logistic regression analysis by calculation of the odds ratio.
Association of the possible condition to the permanence or
not of the pathological group where it has been defined.

| Group | CONDITION | P-value | OR (95% CI) | |
|---|---|---|---|---|
| Control | IGFP1 negative | P < 0.001 | 50 | (5.64-500) |
| (without | MIP1B negative | | | |
| COPD or | CCL1 negative | | | |
| LC) | MIG negative | | | |
| | PDGFAA negative | | | |
| COPD | IGFP1 negative | 0.002 | 12.071 | (2.485-58.629) |
| Adenocar- | CCL1 positive | 0.002 | 20.83 | (7.69-76.92) |
| cinoma | MIP1B negative | | | |
| Squamous | CCL1 negative | 0.009 | 5.88 | (1.52-22.2) |
| carcinoma | EGF negative | | | |
| | PDGFAA or MIP1B | | | |
| | positive | | | |
| Adenocar- | CCL1 positive | 0.001 | 5.31 | (1.96-14.49) |
| cinoma | VEGF negative | | | |
| and COPD | | | | |
| Squamous | GDF 15 positive | 0.002 | 9.34 | (2.19-40) |
| carcinoma | VEGF positive | | | |
| and COPD | CCL-1 negative | | | |
| | EGF negative | | | |

### Example 2

### Additional Validation of Cytokines IL-11 and CCL-1

### Material and Methods

### Patients and Samples

For performing this prospective study, between the years 2009 and 2011a total of 359 patients were selected, that had required flexible bronchoscopy for diagnosis. All bronchoalveolar lavage samples (BAL) were collected from patients of the Hospital Virgen del Rocío (Seville, Spain). The screening criteria for the study were:

1) the patients had been evaluated by pneumology departments for haemoptysis or pulmonary nodule,
2) smokers or former smokers of over 20 packs a year,
3) older than 40 years

The exclusion criteria were:

1) presence of another carcinoma or sarcoma,
2) active pulmonary tuberculosis,
3) previous pulmonary resection,
4) history of drug abuse, and
5) presence of another acute or chronic inflammatory disease, besides the chronic obstructive pulmonary disease (COPD). Approval was obtained for this study from the institutional review board. In addition, according to the committee regulations, informed consent was obtained from the participants.

The protein profiles of BAL of the different patient groups were analysed comparing the control group (patients without COPD or lung cancer) with the groups of patients. The latter include a COPD group, a lung cancer group, and a COPD plus cancer of the lung group. The samples were divided into three cohorts. A first group of samples, of 60 patients, was used for developing the initial study. The description of the patients included in the study is shown in Table 5A. The second group of independent samples of 139 different patients was used (Table 5B) and a third group of independent samples of 160 different patients (Table 5C).

TABLE 5
(A) Characteristics of the control population used for protein array. (B) Characteristics of the population
used for validation. (C) Characteristics of the population used for the additional validation.
A

| | | | | COPD with |
|---|---|---|---|---|
| | Control | COPD | Lung cancer | lung cancer |
| | n = 16 | n = 15 | n = 17 | n = 12 |
| Gender | | | | |

| | | | | | | | | |
|---|---|---|---|---|---|---|---|---|
| Male | 100.0% | (16) | 100.0% | (15) | 100.0% | (17) | 100.0% | (12) |
| Female | 0.0% | (0) | 0.0 | (0) | 0.0% | (0) | 0.0% | (0) |
| Mean age | 61.3 | [41-80] | 61.5 | [45-78] | 60.7 | [46-69] | 60.7 | [49-68] |

At present

| | | | | | | | | |
|---|---|---|---|---|---|---|---|---|
| Smoker | 68.8% | (11) | 53.3% | (8) | 52.9% | (9) | 83.3% | (10) |
| Former smoker | 31.2% | (5) | 46.7% | (7) | 47.1% | (8) | 16.7% | (2) |
| Age ranges | 38.8 | [31-53.2] | 50 | [42-65] | 58.2 | [41-65.7] | 52.3 | [41-63.2] |

COPD

| | | | | | | |
|---|---|---|---|---|---|---|
| Mild | — | 20.0% | (3) | — | 58.3% | (7) |
| Moderate | — | 33.3% | (5) | — | 25.0% | (3) |
| Severe | — | 26.7% | (4) | — | 0.0% | (0) |
| Very severe | — | 20.0% | (3) | — | 16.7% | (2) |

Lung cancer

| | | | | | | |
|---|---|---|---|---|---|---|
| Adenocarcinoma | — | — | 70.6% | (12) | 33.3% | (4) |
| Stage I-II | | | 16.6% | (2) | 0.0% | (0) |
| Stage III-IV | | | 83.4% | (10) | 100% | (4) |
| SCC | — | — | 29.4% | (5) | 66.7% | (8) |
| Stage I-II | | | 20% | (1) | 12.5% | (1) |
| Stage III-IV | | | 80% | (4) | 87.5% | (7) |

B

| | | | | | | | COPD with | |
|---|---|---|---|---|---|---|---|---|
| | Control | | COPD | | Lung cancer | | Lung cancer | |
| | n = 20 | | n = 29 | | n = 40 | | n = 50 | |
| Gender | | | | | | | | |
| Male | 60.0% | (12) | 86.2% | (25) | 82.5% | (33) | 96.0% | (48) |
| Female | 40.0% | (8) | 13.8% | (4) | 17.5% | (7) | 4.0% | (2) |
| Mean age | 52.3 | [42-58] | 65.2 | [51-75] | 61.2 | [48-75] | 64.3 | [48-75] |
| At present | | | | | | | | |
| Smoker | 100.0% | (20) | 51.7% | (15) | 50.0% | (20) | 52.0% | (26) |
| Former smoker | 0.0% | (0) | 48.3% | (14) | 50.0% | (20) | 48.0% | (24) |
| Age ranges | 41.8 | [33-57.2] | 52.8 | [41-69.2] | 53 | [39-73.2] | 57 | [43-75.2] |

| COPD | | | | | | |
|---|---|---|---|---|---|---|
| Mild | — | 27.6 | (8) | — | 26.0% | (13) |
| Moderate | — | 51.7% | (15) | — | 50.0% | (25) |
| Severe | — | 20.7% | (6) | — | 24.0 | (12) |
| Very severe | — | 0.0% | (0) | — | 0.0% | (0) |

| Lung cancer | | | | | | |
|---|---|---|---|---|---|---|
| Adenocarcinoma | — | — | 37.5% | (15) | 46.0% | (23) |
| Stage I-II | | | 26.7% | (4) | 21.7% | (5) |
| Stage III-IV | | | 73.3% | (11) | 78.3% | (18) |
| SCC | — | — | 62.5% | (25) | 54.0% | (27) |
| Stage I-II | | | 40% | (10) | 55.6% | (15) |
| Stage III-IV | | | 60% | (15) | 44.4% | (12) |

C

| | Control | LC | COPD with LC |
|---|---|---|---|
| | n = 20 | n = 66 | n = 74 |
| Gender | | | |

| | | | | | | |
|---|---|---|---|---|---|---|
| Male | 60.0% | (12) | 83.3% | (55) | 94.6% | (70) |
| Female | 40.0% | (8) | 16.7% | (11) | 5.4% | (4) |
| Mean age [range] | 52.3 | [42-58] | 61.2 | [48-75] | 64.3 | [48-75] |
| At present | | | | | | |
| Smoker | 100.0% | (20) | 50.0% | (33) | 52.7% | (39) |
| Former smoker | 0.0% | (0) | 50.0% | (33) | 47.3% | (35) |
| Age ranges | 36.3 | [30-51.2] | 58.8 | [41-77.2] | 62 | [43-79.2] |

| Lung cancer | | | | | |
|---|---|---|---|---|---|
| Adenocarcinoma | — | 22.7% | (15) | 16.2% | (12) |
| Stage I-II | | 20% | (3) | 16.7 | (2) |
| Stage III-IV | | 80% | (12) | 83.3% | (10) |
| SCC | | 18.2% | (12) | 13.5% | (10) |
| Stage I-II | | 33.3% | (4) | 60% | (6) |
| Stage III-IV | | 66.6% | (8) | 30% | (4) |
| LCC | — | 12.1% | (8) | 18.9% | (14) |
| Stage I-II | | 37.5% | (3) | 42.85% | (6) |
| Stage III-IV | | 62.5% | (5) | 57.15% | (8) |
| SCLC | — | 47.0% | (31) | 51.4% | (38) |
| Limited stage | | 41.9% | (13) | 42.1% | (16) |
| Extensive stage | | 58.1% | (18) | 57.9% | (22) |

The subjects were treated with a combination of topical anaesthesia (20% benzocaine aerosol into the pharynx plus 2% topical lidocaine on demand) and conscious sedation using midazolam and meperidine according to the institutional guidelines. Bronchoalveolar lavage samples were obtained by instillation and aspiration in the bronchopulmonary segment of 40-60 mL aliquots of sterile saline solution. The fluid recovered was immediately run through a sterile 100 μm nylon filter (Becton Dickinson, San Jose, Calif.) to clear mucus and was transported in ice to the laboratory. The total volume was centrifuged at 4° C. for 10 minutes at 1800×g and frozen at −80° C. until subsequent use.

### Handling of the Sample

About 4-8 mL of BAL sample were used in our tests. Due to their low protein contents, the BAL samples needed to be concentrated before use. The BAL samples were unfrozen in ice with a protease inhibitor kit (Thermo Scientific, Franklin, Mass., US). The samples were aliquoted in new tubes and placed in a vacuum concentrate (Concentrator plus—Eppendorf, Hamburg, Germany). The initial volume of the samples was reduced from 1.5 to 2 mL in 2-6 hours. The quantitation of proteins was performed by the RCDC method (Bio-Rad, Hercules, Calif., US).

### Protein Array

For the purpose of studying the protein profiles of the four patient groups, a screening was performed for cytokines and growth factor. The protein matrix used in the study is available in the market and analyses the expression levels of 80 cytokines and growth factors (Quantibody® Antibody cytokines Humans Array 1000—RayBiotech, Norcross, Ga., US). Tables 6 and 7 contain a full list of the proteins analysed in the study. The test matrix for human cytokines was performed according to the instructions provided by the manufacturer. The intensity of each signal was viewed by using a scanner laser model GenePix 4100 A (Molecular Devices, Sunnyvale, Calif., US). The intensity points of each cytokine (four spots per protein/positive control) were melted and expressed as the mean relative to the average signals of the positive controls in the microarrays analysed for the groups of diseases (patients with COPD, with lung cancer, with COPD and with lung cancer) and the control group (patients without COPD or lung cancer).

TABLE 6
Characteristics of the antibodies used in the study

| Cytokine symbol | Cytokine name |
|---|---|
| BLC | B-cell lymphoma |
| Eotaxin | Eoxtaxin |
| Eotaxin 2 | Eotaxin 2 |
| G-CSF | Granulocyte colony stimulating factor |
| GM-CSF | Granulocyte macrophage colony stimulating factor |
| I-309/CCL1 | Chemokine (C-C motif) ligand 1 |
| ICAM 1 | Intercellular adhesion molecule 1 |
| IFN g | Interferon-gamma |
| IL-1a | Interleukin-1 alpha |
| IL-1b | Interleukin-1 beta |
| IL-1ra | Interleukin receptor antagonist 1 |
| IL-2 | Interleukin-2 |
| IL-4 | Inerleukin-4 |
| IL-5 | Interleukin-5 |
| IL-6 | Interleukin 6 |
| IL-6Sr | Interleukin-6 soluble receptor |
| IL-7 | Interleukin-7 |
| IL-8 | Interleukin-8 |
| IL-10 | Interleukin-10 |
| IL-11 | Interleukin-11 |
| IL-12p40 | Interleukin-12 p40 |
| IL-12p70 | Interleukin-12 p70 |
| IL-13 | Interleukin-13 |
| IL-15 | Interleukin-15 |
| IL-16 | Interleukin-16 |
| IL-17 | Interleukin-17 |
| MCP-1/CCL2 | Monocyte chemotactic protein 1/chemokine |
| | (C-C motif) ligand 2 |
| MCSF | Mouse stem cell factor |
| MIG/CXCL9 | Monokine induced by IFN-Gamma/Chemokine |
| | (C-X-C motif) ligand 9 |
| MIP 1 B-1a/CCL3 | Macrophage Inflammatory Protein 1 alpha/ |
| | Chemokine (C-C motif) ligand 3 |
| MIP 1 B-1b/CCL4 | Macrophage Inflammatory Protein 1 beta/ |
| | Chemokine (C-C motif) ligand 4 |
| MIP-1 B-1d | Macrophage Inflammatory Protein 1 delta |
| PDGF-BB | BB platelet-derived growth factor |
| Rantes/CCL5 | Regulated on normal activation of T cells |
| | expressed, and secreted/Chemokine (C-C motif) |
| | ligand 5 |
| TIMP-1 | Tissue inhibitor of metalloproteinases 1 |
| TIMP-2 | Tissue inhibitor of metalloproteinases 2 |
| TNF a | Tumour necrosis factor alpha |
| TNF b | Tumour necrosis factor beta |
| TNF RI | Tumour necrosis factor 1 |
| TNF RII | Tumour necrosis factor 2 |

TABLE 7
Characteristics of the antibodies used in the study

| Growth factor | |
|---|---|
| symbol | Growth factor name |
| AR | Androgen receptor |
| BNDF | Brain neutrophil derived factor |
| b-FGF | Basic fibroblast growth factor |
| BMP4 | Bone morphogenetic protein 4 |
| BMP5 | Bone morphogenetic protein 5 |
| BMP7 | Bone morphogenetic protein 7 |
| B-NGF | Beta nervous growth factor |
| EGF | Epidermal growth factor |
| EGFR | Epidermal growth factor receptor |
| EG-VEGF | Endocrine gland derived vascular |
| | endothelial growth factor |
| FGF 4 | Fibroblast growth factor 4 |
| FGF 7 | Fibroblast growth factor 7 |
| GDF-15 | Growth differentiation factor 15 |
| GDNF | Glial cell derived neutrophil factor |
| GH | Growth hormone |
| HB-EGF | Heparin-binding EGF-like growth factor |
| HGF | Hepatocyte growth factor |
| IGFBP-1 | Insulin-like growth factor binding protein 1 |
| IGFBP-2 | Insulin-like growth factor binding protein 2 |
| IGFBP-3 | Insulin-like growth factor binding protein 3 |
| IGFBP-4 | Insulin-like growth factor binding protein 4 |
| IGFBP-6 | Insulin-like growth factor binding protein 6 |
| IGF-1 | Insulin-like growth factor 1 |
| Insulin | Insulin |
| MCF R | Macrophage chemotactic factor receptor |
| NGF R | Nervous growth factor receptor |
| NT-3 | Neurotrophin 3 |
| NT-4 | Neurotrophin 4 |
| OPG | Osteoprotegerin |
| PDGFAA | Platelet-derived growth factor AA |
| PIGF | Phosphatidylinositol-glycan biosynthesis class F |
| SCF | Stem cell factor |
| SCF R | Stem cell factor receptor |
| TFG a | Transforming growth factor alpha |
| TGF b1 | Transforming growth factor beta 1 |
| TGF b3 | Transforming growth factor beta 3 |
| VEGF | Vascular endothelial growth factor |
| VEGF R2 | Vascular endothelial growth factor receptor 2 |
| VEGF R3 | Vascular endothelial growth factor receptor 3 |
| VEGF D | Vascular endothelial growth factor D |

### Western Blot

BAL proteins (50 μg) were separated as described above. In summary, the SDS-PAGEs were transferred to PVDF membranes (Bio-Rad, Hercules, Calif., US). After blocking, the blots were incubated with the following primary antibodies: anti-IL11 and anti-CCL-1 (1:200 Abcam, Cambridge, Mass., US). The secondary antibodies used were: peroxidase conjugated with anti-mouse (GE Healthcare, Uppsala, Sweden) and anti-rabbit (Cell signalling, Beverly, Mass., US). The protein bands were revealed using an increase of ECL chemoluminiscence (GE Healthcare, Uppsala, Sweden) and viewed in an image analyser (Mini LAS-3000, Fujifilm, Tokyo, Japan). The quantification of the expression levels was performed by comparison to the quantity of β-actin protein (1:1000 Abcam, Cambridge, Mass., US). To analyse the expression values of the proteins of interest obtained by Western blot densitometry was used. The densitometry analysis of the blots scanned was performed using the software Image J (http://rsbweb.nih.gov/ij/) and the results were expressed as the relative changes from the control protein (β-actin). The tests were repeated three times independently.

### ELISA

The BAL samples were evaluated according to the manufacturer's instructions, using sandwich ELISA tests for CCL-1/I-309 and IL-11 (DuoSet, R % D Systems, Minneapolis, Minn., US). All samples were tested twice and using the same plate. In summary, the antibody is captured (concentration provided by the manufacturer) was diluted in PBS, added to each well and allowed all night long at 4° C. The plate was washed four times in TBS with 0.05% of Tween-20 (TBST 0.05%). The plate was blocked with 1% of bovine serum albumin (BSA) for 1 hour at room temperature before washing it again four times with 0.05% TBST. The samples and the standards were added to the plate and incubated for 2 hours at room temperature. Then the plate was wash, the detection antibody was added (concentration provided by the manufacturer) diluted in PBS. The plate was incubated for 2 hours at room temperature. Once the plate was washed again, streptavidine was added (DuoSet, R & D Systems) and the plate was incubated for 30 minutes. Finally, colour reagent o-phenylenediamine was added to each well and the reaction was allowed to developed in darkness for 20 minutes. The reaction was stopped by adding 2 N H₂SO₄ to each well. Absorbance was read in a plate reader at 450 nm (Emax, Molecular Devices, Minneapolis, Minn., US).

### Statistical Analysis

All continuous variables of the characteristics of the patients were expressed as median for each variable (interquartile range [IQR]) and the categorical variables as the number of cases and percentage. The statistical analysis was performed by the statistical package for Social Sciences (SPSS 17, Chicago, Ill., US).

### Analysis of Protein Array Expression Data

The analysis of hierarchical grouping was performed using the function UPGMA (Unweighted Pair Group Method with Arithmetic Mean). Before the statistical analysis, the protein expression levels were standardised protein by protein under all conditions, using the means and SD values. The sample conditions were grouped using Euclidian distance metric tests. The results were viewed and analysed with Babelomics 4.2 (babelomics.bioino.cipf.es). the expression level of each protein with respect to its mean level of expression under all conditions was represented by a colour, with red represents a greater expression than the median, blue represents a lower expression than the median, and several intermediate colour intensities that represent the magnitude of the variance from the median.

### Analysis of the Validity of the Diagnostic Tests

The differences between the two independent groups were analysed with a Mann-Whitney U test (continuous variables and non-parametric tests). The ROC curves were built to evaluate sensitivity, specificity and the respective areas under the curve (AUC) with 95% CI. The optimum cut off value was investigated that was chosen to show the best combination between sensitivity, specificity, positive predictive value, negative predictive value, odds ratio to predict diagnostic adenocarcinoma. To test the accuracy of the diagnosis when measuring both IL-11 and CCL-1, functions of markers combined by binary logistic regression were estimated.

### Results

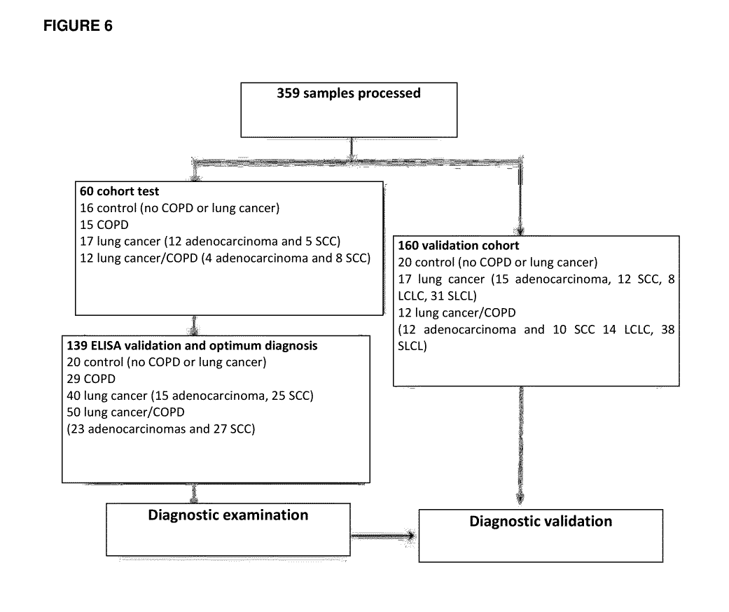

Overall 359 patients were recruited, 60 in the screening cohort, 139 in the validation cohort, and 160 in the additional validation cohort. The clinical-pathological characteristics of the patients in the study and the validation cohorts are summarised in FIG. 6.

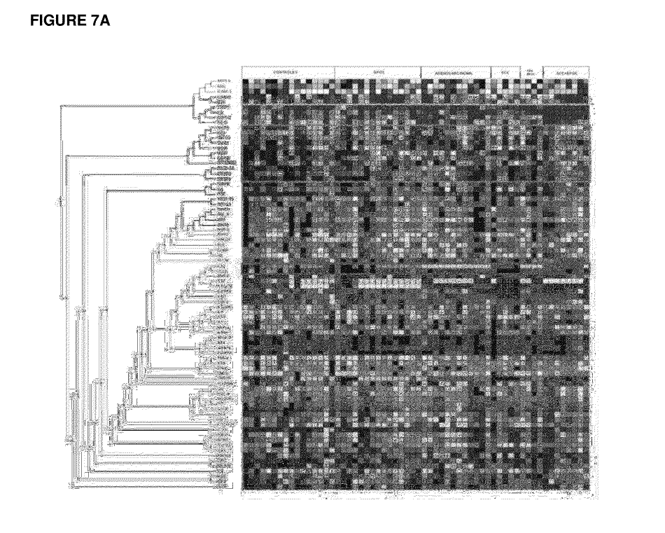

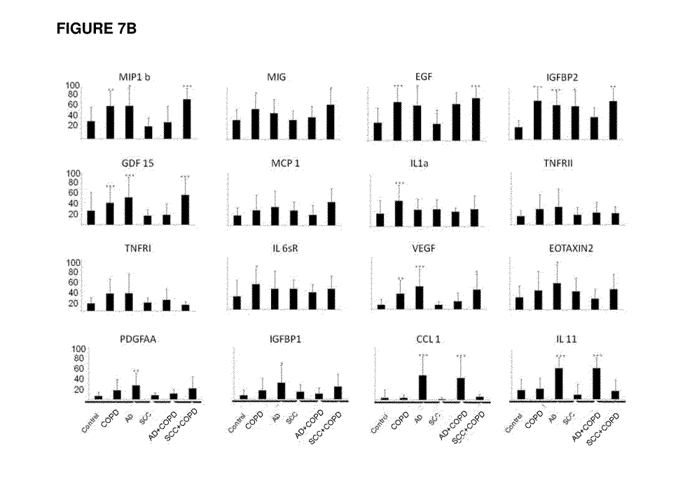

First, the expression profiles of inflammatory proteins were analysed using expression arrays, using the BAL samples of patients of test cohort. Characteristics of the patients were distributed homogeneously into four different groups: COPD, LC, both diseases or none (control group). The analysis disclosed that a significant number (16) of proteins (MIP1b, MIG, IGFBP2, IGFBP1, EGF, VEGF, TNFR-I, TNFR-Ii, IL6sR, GDF15, IL-1a, MCP-1, EOTAXIN2, PDGFAA, IL-11, and CCL-1) participating in the airways were overexpressed in the patient groups with lung cancer and/or COPD as compared to the control group. Surprisingly, there were no clear differences in protein expression between the histological subtype of adenocarcinoma and the rest of the other groups. More specifically, the expression of IL-11 and CCL1 appeared only in the adenocarcinoma condition (FIG. 7A).

A formal statistical analysis (FIG. 7B) confirmed the results previously obtained in the heat modelling map despite the dispersion existing in the groups tested. In addition, CCL-1 and proteins IL-11 evidence statistically significant expression differences in the patients with adenocarcinoma as compared to the other groups of patients.

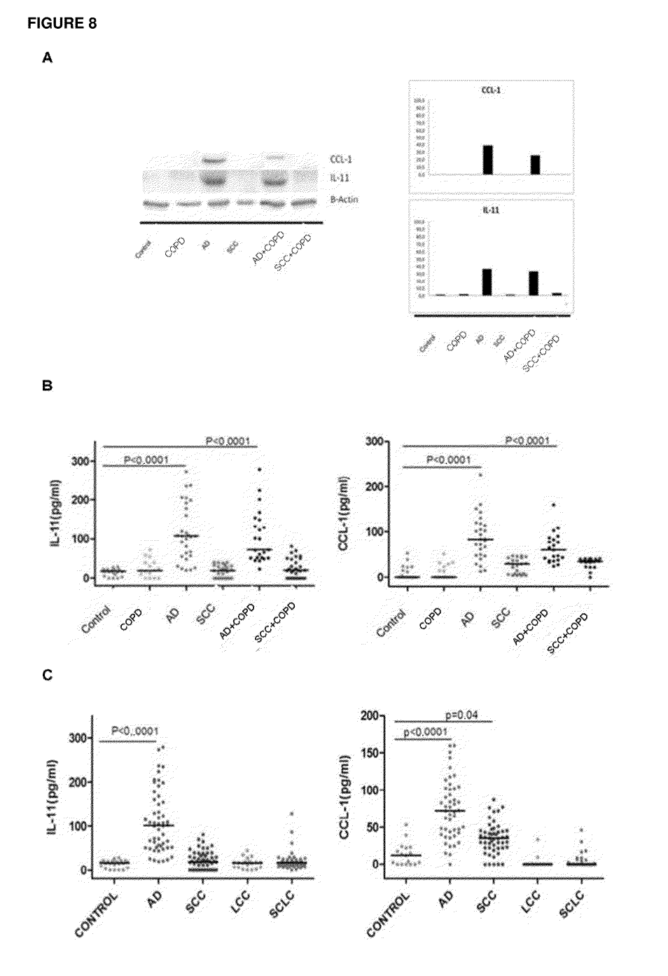

For the purpose of validating the results seen in the protein arrays, a Western blot of proteins associated only with the groups of adenocarcinoma of IL-11 and CCL-1 was performed (FIG. 8A). the results of the Western blot tests indicated differences of expression similar to those found in the analysis of the heat map.

To analyse the reliability of the profile obtained the proteins previously identified by ELISA method were validated individually. This method was chosen for several reasons: validating the results with a different method, its high sensitivity and its easy handling and clinical application. The expression of IL-11 and CCL-1 was analysed in two controlled independent cohorts. In the first cohort of validation, the 139 patients (Table 6) had clinical-pathological characteristics similar to the cohorts of the test. On the other hand, the additional validation cohort of 160 patients (Table 7) differed from the study cohort, given the inclusion of other histological subtypes of lung cancer and the absence of a COPD group. The results evidenced that IL-11 and CCL-1 were significantly overexpressed in patients with adenocarcinoma as compared to the other groups, in the first validation cohort (FIG. 8B) and in the additional validation cohort (FIG. 8C).

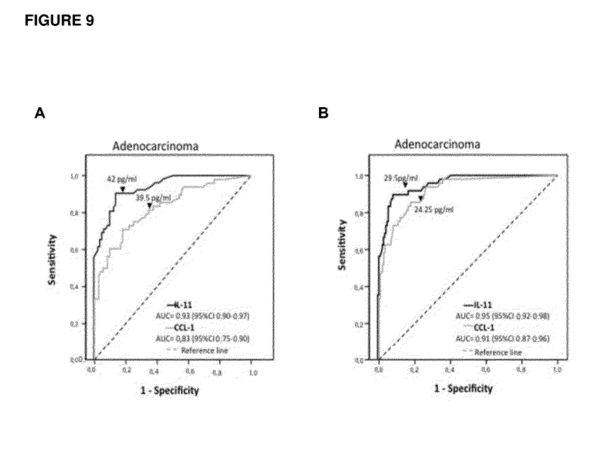

Subsequently, the diagnostic value of both proteins for adenocarcinoma of the lung was analysed by ROC curves for the purpose of obtaining a cut-off line which allow to classify the patients as adenocarcinoma or no adenocarcinoma (FIG. 9).

The ROC curves for validation cohorts evidenced that the optimum cut-off value for IL-11 was 42 pg/mL (AUC: 0.935, 95% CI: 0.896-0.75), with a 90% sensitivity and a specificity of 86% (FIG. 8A, Table 5A). The optimum cut-off value for CCL-1 was 39.5 pg/mL (AUC: 0.83, 95% CI: 0.749-0.902), with a sensitivity of 83% and a specificity of 74% (FIG. 8A, Table 5A). In addition, the sensitivity and specificity of proteins CCL-1 and IL-11 overall and CCL-1 and/or IL-11 was analysed (Table 8A). On the other hand, the ROC curves in the additional validation cohort evidence that the optimum cut-off value for the diagnosis IL-11 was 29.5 pg/mL (AUC: 0.95, 95% CI: 0.92-0.98), with a sensitivity of 90.6% and a specificity of 83% (FIG. 8 B, Table 1B). the optimum cut-off value for CCL-1 was 24.25 pg/mL (AUC: 0.91, 95% CI: 0.87-0.96), with a sensitivity of 91.7%, and a specificity of 77.% (FIG. 8B, Table 1 b). In addition, the sensitivity and specificity of proteins CCL-1 and IL-11 overall and CCL-1 and/or IL-11 were analysed for this cohort (Table 8B).

TABLE 8

| | | | | | | |
|---|---|---|---|---|---|---|
| AUC | Sensitivity | Specificity | PPV | NPV | Positive | Negative |
| (95% CI) | (95% CI) | (95% CI) | (95% CI) | (95% CI) | LR | LR |

A. Cohort validation test

| | | | | | | | |
|---|---|---|---|---|---|---|---|
| IL-11 | 0.930 | 90.2% | 88.7% | 80.7% | 94.5% | 7.95 | 0.11 |
| | (0.896-0.975) | (79.0-95.7%) | (80.6-93.5%) | (68.7-88.9%) | (87.8-97.6%) | (4.53-13.98) | (0.05-0.26) |
| CCL-1 | 0.830 | 80.0% | 74.1% | 72.1% | 86.3% | 3.02 | 0.29 |
| | (0.794-0.902) | (66.4-87.7%) | (63.9-82.2%) | (59.2-73.4%) | (76.6-92.4%) | (2.05-4.47) | (0.17-0.52) |
| IL-11 | | 71.2% | 94.4% | 86.0% | 87.2% | 12.80 | 0.31 |
| and | | (57.7-81.7%) | (88.4-97.4%) | (72.7-93.4%) | (79.9-92.1%) | (5.77-28.41) | (0.20-0.47) |
| CCL-1 | | | | | | | |
| IL-11 | | 94.3% | 74.1% | 64.1% | 96.4% | 3.64 | 0.08 |
| and/or | | (84.6-98.1%) | (65.1-81.4%) | (53.0-73.9%) | (89.9-98.8%) | (2.63-5.04) | (0.03-0.23) |
| CCL-1 | | | | | | | |

B. Validation test of the additional cohort

| | | | | | | | |
|---|---|---|---|---|---|---|---|
| IL-11 | 0.95 | 90.6% | 83.0% | 60.8% | 96.8% | 5.32 | 0.11 |
| | (0.92-0.98) | (79.7-95.9%) | (86.8-87.7%) | (49.7-70.8%) | (92.7-98.6%) | (3.81-7.41) | (0.05-0.26) |
| CCL-1 | 0.91 | 91.7% | 77.5% | 51.2% | 97.3% | 4.08 | 0.11 |
| | (0.87-0.96) | (80.4-96.7%) | (71.0-82.9%) | (40.8-61.4%) | (93.3-99.0%) | (3.09-5.04) | (0.04-0.28) |
| IL-11 | | 71.2% | 96.3% | 84.1% | 92.3% | 19.10 | 0.30 |
| and | | (57.7-81.7%) | (92.5-98.2%) | (70.6-92.1%) | (87.7-95.3%) | (9.00-41.13) | (0.19-0.46) |
| CCL-1 | | | | | | | |
| IL-11 | | 92.3% | 84.0% | 62.5% | 98.1% | 5.88 | 0.07 |
| and/or | | (82.6-98.1%) | (78.0-88.5%) | (51.5-72.3%) | (94.6-99.4%) | (4.21-8.22) | (0.02-0.20) |
| CCL-1 | | | | | | | |

C. Validation of the first cohort in the second cohort

| | Sensitivity | Specificity | PPV | NPV | Positive | Negative |
|---|---|---|---|---|---|---|
| | (95% CI) | (95% CI) | (95% CI) | (95% CI) | LR | LR |
| IL-11 | 90.2% | 91.4% | 75.4% | 97.0% | 10.52 | 0.11 |
| | (79.7-95.9%) | (86.3-94.7%) | (63.3-84.5%) | (93.1-98.7%) | (6.43-17.22) | (0.05-0.25) |
| CCL-1 | 78.3% | 85.4% | 61.0% | 93.1 | 5.38 | 0.25 |
| | (64.4-87.7%) | (79.1-90.1%) | (48.3-72.4%) | (87.8-96.2%) | (3.58-8.08) | (0.15-0.44) |
| IL-11 and | 71.2% | 96.6% | 86.0% | 91.9% | 20.87 | 0.30 |
| CCL-1 | (57.7-81.7%) | (92.8-98.4%) | (72.7-93.4%) | (87.1-95.0%) | (9.33-46.69) | (0.19-0.46) |
| IL-11 and/or | 93.0% | 77.8% | 51.9% | 97.7% | 4.20 | 0.09 |
| CCL-1 | (81.4-97.6%) | (71.0-83.5%) | (41.0-62.7%) | (93.6-99.2%) | (3.12-5.64) | (0.03-0.27) |

AUC = area under the curve.
PPV = positive predictive value.
NPV = negative predictive value.
LR = likelihood ratio

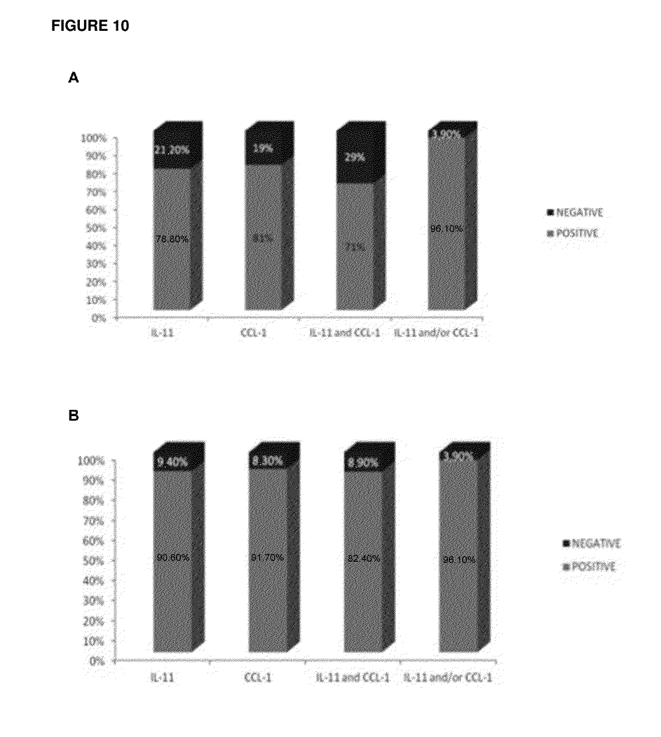

As sensitivity and specificity were similar in both groups, a binary logistic regression was performed to analyse the validity of the cut-off values obtained in the previous analysis. The cut-off value obtained in the first validation cohort had a diagnostic capacity for adenocarcinoma of 78.8% and 81% of the patients with IL-11 and CCL-1, respectively (FIG. 10A). This capacity increased to 90% and 91% in patients with IL-11 and CCL-1, respectively, in the additional validation cohort, detecting up to 96% of the patients when both biomarkers where used (FIG. 10B). 40 pg/mL and 39.5 pg/mL were used as cut-off value for adenocarcinoma. The predictive values and odds ratios in the diagnosis of adenocarcinoma are given in Table 9. In parallel the differential diagnostic precision was evaluated, joining two validation cohorts. Subsequently, the AUC, sensitivity, specificity, PPV, NPV and the relationship between initial stages, former smokers, packs smoked/year, number of cells in BAL, and involvement of the bronchial tract were analysed (Table 9). The ROC analyses evidenced that the test for IL-11 and CCL-1 increased the accuracy of the diagnosis of adenocarcinoma in early stage, the AUC of IL-11 was 0.95 (95% CI: 0.91-0.99) with a sensitivity of 100% and specificity of 93%, and the AUC for CCL-1 was 0.93 (95% CI: 0.85-1) with a sensitivity of 91.7% and a specificity of 81.6% (table 9). It was also seen that the AUC, sensitivity and specificity of both proteins in adenocarcinoma was increased in former smokers and with the packs smoked per year f (>30 p; Table 9B, 9C).

TABLE 9

| | | | | | | |
|---|---|---|---|---|---|---|
| AUC | Sensitivity | Specificity | PPV | NPV | Positive | Negative |
| (95% CI) | (95% CI) | (95% CI) | (95% CI) | (95% CI) | LR | LR |

A. Early stages

| | | | | | | | |
|---|---|---|---|---|---|---|---|
| IL-11 | 0.95 | 100.0% | 93.0% | 87.0% | 100.0% | 14.33 | 0 |
| | (0.91-0.99) | (83.9-100.0%) | (81.4-97.6%) | (67.9-95.5%) | (91.2-100.0%) | (4.81-42.69) | |
| CCL-1 | 0.93 | 91.7% | 81.6% | 61.1% | 96.9% | 4.98 | 0.10 |
| | (0.85-1.00) | (64.6-98.5%) | (66.6-90.8%) | (38.6-79.7%) | (84.3-99.4%) | (2.49-9.93) | (0.02-0.68) |
| IL-11 | | 76.9% | 95.3% | 83.3% | 93.2% | 16.54 | 0.24 |
| and | | (49.7-91.8%) | (84.5-98.7%) | (55.2-95.3%) | (81.8-97.7%) | (4.14-66.11) | (0.09-0.66) |
| CCL-1 | | | | | | | |
| IL-11 | | 100.0% | 69.8% | 50.0% | 100.0% | 3.31 | 0 |
| and/or | | (77.2-100.0%) | (54.9-81.4%) | (32.1-67.9%) | (88.6-100.0%) | (2.10-5.21) | |
| CCL-1 | | | | | | | |

B. Former smokers

| | | | | | | | |
|---|---|---|---|---|---|---|---|
| IL-11 | 0.97 | 100.0% | 93.2% | 77.3% | 100.0% | 14.80 | 0 |
| | (0.95-1.00) | (81.6-100.0%) | (85.1-97.1%) | (56.6-89.9%) | (94.7-100.0%) | (6.35-35.40) | |
| CCL-1 | 0.97 | 88.9% | 87.8% | 64.0% | 97.0% | 7.31 | 0.13 |
| | (0.94-1.00) | (67.2-96.2%) | (78.5-93.5%) | (44.5-79.8%) | (89-8-99.2%) | (3.88-13.77) | (0.03-0.47) |
| IL-11 | | 94.1% | 92.6% | 80.0% | 98.0% | 12.71 | 0.06 |
| and | | (73.0-99.0%) | (82.4-97.1%) | (58.4-91.9%) | (89.7-99.7%) | (4.91-32.87) | (0.01-0.43) |
| CCL-1 | | | | | | | |
| IL-11 | | 100.0% | 85.0% | 58.6% | 100.0% | 6.67 | 0 |
| and/or | | (81.6-100.0%) | (75.6-91.2%) | (40.7-75.4%) | (94.7-100.0%) | (3.96-11.23) | |
| CCL-1 | | | | | | | |

C. Smokers of less than 30 packs a year

| | | | | | | | |
|---|---|---|---|---|---|---|---|
| IL-11 | 0.94 | 89.5% | 96.8% | 94.4% | 93.8% | 27.74 | 0.11 |
| | (0.90-0.98) | (68.6-97.1%) | (83.8-99.4%) | (74.2-99.0%) | (79.9-98.3%) | (4.01-191.91) | (0.03-0.40) |
| CCL-1 | 0.90 | 92.3% | 96.6% | 92.3% | 96.6% | 26.77 | 0.08 |
| | (0.84-0.97) | (66.7-96.6%) | 82.8-99.4%) | 66.7-98.6%) | 82.8-99.4%) | (3.88-184.85) | (0.01-0.53) |
| IL-11 | | 92.3% | 95.8% | 92.3% | 95.8% | 22.15 | 0.08 |
| and | | (66.7-98.6%) | (79.8-99.3%) | (66.7-98.6%) | (79.8-99.3%) | (3.23-15.189) | (0.01-0.53) |
| CCL-1 | | | | | | | |
| IL-11 | | 100.0% | 83.9% | 72.2% | 100.0% | 6.20 | 0 |
| and/or | | (77.2-100.0%) | 67.4-92.9%) | (49.1-87.5%) | (87.1-100.0%) | (2.78-13.84) | |
| CCL-1 | | | | | | | |

The comparative analyses of the number of cells in BAL of patients with adenocarcinoma evidenced that patients with a lower number of cells than the median had similar AUC, sensitivity and specificity results with the patients above the median number of cells (Table 10).

TABLE 10

| | | | | | | |
|---|---|---|---|---|---|---|
| AUC | Sensitivity | Specificity | PPV | NPV | Positive | Negative |
| (95% CI) | (95% CI) | (95% CI) | (95% CI) | (95% CI) | LR | LR |

A. Number of cells per million above the median

| | | | | | | | |
|---|---|---|---|---|---|---|---|
| IL-11 | 0.90 | 91.7% | 95.5% | 91.7% | 95.5% | 20.17 | 0.09 |
| | (0.83-0.96) | (74.2-97.7%) | (84.9-98.7%) | (74.2-97.7%) | (84.9-98.7%) | (5.18-78.53) | (0.02-0.13) |
| CCL-1 | 0.91 | 86.4% | 85.7% | 70.4% | 94.1% | 6.05 | 0.16 |
| | (0.86-0.96) | (66.7-95.3%) | (74.3-92.6%) | (51.5-84.1%) | (84.1-98.0%) | (3.12-11.73) | (0.05-0.46) |
| IL-11 | | 87.0% | 100.0% | 100.0% | 94.1% | 0 | 0.13 |
| and | | (67.9-95.5%) | (92.6-100.0%) | (83.9-100.0%) | (84.0-98.0%) | | (0.05-0.37) |
| CCL-1 | | | | | | | |
| IL-11 | | 100.0% | 84.9% | 75.0% | 100.0% | 6.63 | 0 |
| and/or | | (86.2-100.0%) | (72.9-92.1%) | (57.9-86.7%) | (92.1-100.0%) | (3.50-12.55) | |
| CCL-1 | | | | | | | |

B. Number of cells per million above the median

| | | | | | | | |
|---|---|---|---|---|---|---|---|
| IL-11 | 0.90 | 86.4% | 86.8% | 73.1% | 93.9% | 6.54 | 0.16 |
| | (0.84-0.98) | (66.7-95.3%) | (75.2-93.5%) | (53.9-86.3%) | (83.5-97.9%) | (3.22-13.30) | (0.05-0.45) |
| CCL-1 | 0.91 | 75.0% | 82.9% | 71.4% | 85.3% | 4.38 | 0.30 |
| | (0.85-0.96) | (53.1-88.8%) | (67.3-91.9%) | (50.0-86.2%) | (69.9-93.6%) | (2.02-9.46) | (0.14-0.66) |
| IL-11 | | 80.0% | 98.1% | 94.1% | 92.7% | 41.60 | 0.20 |
| and | | (58.4-91.9%) | (89.9-99.7%) | (73.0-99.0%) | (82.7-97.1%) | (5.90-293.39) | (0.08-0.49) |
| CCL-1 | | | | | | | |
| IL-11 | | 90.5% | 73.6% | 57.6% | 95.1% | 3.43 | 0.13 |
| and/or | | (71.1-97.3%) | (60.4-83.6%) | (40.8-72.8%) | (83.9-98.7%) | (2.14-5.84) | (0.03-0.49) |
| CCL-1 | | | | | | | |

With regard to the tumour location, there were no differences among the patients. Patients with adenocarcinoma of proximal location show a better AUC, sensitivity and specificity than patients with a distal location. The AUC for IL-11 in patients with proximal location was 0.98 (95% CI: 0.96-1) with a sensitivity of 100% and specificity of 91.4%, while the AUC for IL-11 for patients with distal location was 0.92 (95% CI: 0.87-0.94), with a sensitivity of 89.3% and a specificity of 91.5%. Similar results have been obtained for CCL-1 (Table 11).

TABLE 11

| | | | | | | |
|---|---|---|---|---|---|---|
| AUC | Sensitivity | Specificity | PPV | NPV | Positive | Negative |
| (95% CI) | (95% CI) | (95% CI) | (95% CI) | (95% CI) | LR | LR |

A. Proximal location

| | | | | | | | |
|---|---|---|---|---|---|---|---|
| IL-11 | 0.92 | 100.0% | 91.4% | 60.5% | 100.0% | 11.60 | 0 |
| | (0.88-0.94) | (85.7-100.0%) | (86.3-94.7%) | (44.7-74.4%) | (97.6-100.0%) | (7.15-18.82) | |
| CCL-1 | 0.89 | 100.0% | 82.4% | 50.0% | 100.0% | 5.70 | 0 |
| | (0.83-0.96) | (85.7-100.0%) | (75.0-88.0%) | (36.1-63.9%) | (96.6-100.0%) | (3.93-8.25) | |
| IL-11 | | 82.6% | 96.2% | 73.1% | 97.8% | 21.95 | 0.18 |
| and | | (62.9-93.0%) | (92.4-98.2%) | (53.9-86.3%) | (94.5-99.1%) | (10.36-46.9) | (0.17-0.44) |
| CCL-1 | | | | | | | |
| IL-11 | | 100.0% | 78.5% | 36.5% | 100.0% | 4.65 | 0 |
| and/or | | (85.7-100.0%) | (72.0-88.8%) | (25.7-48.9%) | (97.4-100.0%) | (3.53-12.00) | |
| CCL-1 | | | | | | | |

B. Distal location

| | | | | | | | |
|---|---|---|---|---|---|---|---|
| IL-11 | 0.98 | 89.3% | 91.4% | 62.5% | 98.1% | 10.36 | 0.12 |
| | (0.96-1.00) | (72.8-96.3%) | (86.3-94.7%) | (47.0-66.3%) | (94.7-99.4%) | (6.28-17.08) | (0.04-0.37) |
| CCL-1 | 0.94 | 89.7% | 82.4% | 53.1% | 97.3% | 5.11 | 0.13 |
| | (0.88-0.98) | (73.6-96.4%) | (75.0-88.0%) | (39.4-66.3%) | (92.4-99.1%) | (3.45-7.55) | (0.04-0.37) |
| IL-11 | | 67.9% | 96.2% | 73.1% | 95.2% | 18.03 | 0.33 |
| and | | (49.3-82.1%) | (92.4-98.2%) | (53.9-86.2%) | (91.2-97.5%) | (8.35-38.95) | (0.19-0.58) |
| CCL-1 | | | | | | | |
| IL-11 | | 92.6% | 78.5% | 38.5% | 98.6% | 4.32 | 0.909 |
| and/or | | (76.6-97.9%) | (72.0-83.8%) | (27.6-50.6%) | (95.2-99.6%) | (3.25-5.78) | (0.02-0.36) |
| CCL-1 | | | | | | | |

Finally, the direct and indirect and no change signal was analysed in patients with adenocarcinoma. The diagnostic accuracy in these groups did not show significant differences in the AUC, sensitivity and specificity thereof (Table 12).

TABLE 12

| | | | | | | |
|---|---|---|---|---|---|---|
| AUC | Sensitivity | Specificity | PPV | NPV | Positive | Negative |
| (95% CI) | (95% CI) | (95% CI) | (95% CI) | (95% CI) | LR | LR |

A. Direct signal

| | | | | | | | |
|---|---|---|---|---|---|---|---|
| IL-11 | 0.94 | 93.3% | 91.4% | 48.3% | 99.4% | 10.83 | 0.07 |
| | (0.89-0.99) | (70.2-98.8%) | (86.3-94.7%) | (31.4-65.6%) | (96.5-99.9%) | (6.55-17.89) | (0.01-0.49) |
| CCL-1 | 0.90 | 81.8% | 82.4% | 28.1% | 98.2% | 4.66 | 0.22 |
| | (0.84-0.96) | (52.3-94.9) | (75.0-88.0%) | (15.6-45.4%) | (93.6-99.5%) | (2.93-7.41) | (0.06-0.78) |
| IL-11 | | 62.5% | 96.2% | 58.8% | 96.8% | 16.61 | 0.39 |
| and | | (38.6-81.5%) | (92.4-98.2%) | (36.0-78.4%) | (93.1-98.5%) | (7.32-37.70) | (0.21-0.74) |
| CCL-1 | | | | | | | |
| IL-11 | | 100.0% | 78.5% | 23.1 | 98.6% | 3.99 | 0.18 |
| and/or | | (80.6-100%) | (72.0-83.8%) | (13.7-36.01%) | (95.2-99.6%) | (2.81-5.64) | (0.05-0.66) |
| CCL-1 | | | | | | | |

B. Indirect signal

| | | | | | | | |
|---|---|---|---|---|---|---|---|
| IL-11 | 0.95 | 85.7% | 91.4% | 44.4% | 98.8% | 9.94 | 0.16 |
| | (0.89-0.99) | (60.1-96.0%) | (86.3-94.7%) | (27.6-62.7%) | (95.6-99.7%) | (5.86-16.87) | (0.04-0.57) |
| CCL-1 | 0.89 | 78.6% | 82.4% | 32.4% | 97.3% | 4.48 | 0.26 |
| | (0.78-1.00) | (52.4-92.4%) | (75.0-88.0%) | (19.1-49.2%) | (92.4-99.1%) | (2.82-7.10) | (0.09-0.72) |
| IL-11 | | 80.0% | 96.2% | 63.2% | 98.4% | 21.26 | 0.21 |
| and | | (54.8-93.0%) | (92.4-98.2%) | (41.0-80.9%) | (95.3-94.4%) | (9.85-45.89) | (0.08-0.57) |
| CCL-1 | | | | | | | |
| IL-11 | | 85.7% | 78.5% | 23.1% | 98.6% | 3.99 | 0.18 |
| and/or | | (60.1-96.0%) | (72.0-83.8%) | (13.7-36.1%) | (95.2-99.6%) | (2.81-5.64) | (0.05-0.66) |
| CCL-1 | | | | | | | |

C. Signal without abnormalities

| | | | | | | | |
|---|---|---|---|---|---|---|---|
| IL-11 | 0.98 | 95.0% | 91.4% | 55.9% | 99.4% | 11.02 | 0.05 |
| | (0.95-1.00) | (76.4-99.1%) | (86.3-94.7) | (39.5-71.1%) | (96.5-99.9%) | (6.72-18.03) | (0.01-0.37) |
| CCL-1 | 0.93 | 81.0% | 82.4% | 42.5% | 96.4% | 4.61 | 0.23 |
| | (0.88-0.98) | (60.0-92.3%) | (75.0-88.0%) | (28.5-57.8%) | (91.2-98.6%) | (3.01-7.05) | (0.09-0.56) |
| IL-11 | | 85.0% | 96.2% | 70.8% | 98.4% | 22.59 | 0.16 |
| and | | (64.0-94.8%) | (92.4-98.2%) | (50.8-85.1%) | (95.3-99.4%) | (10.67-24.78) | (0.05-0.44) |
| CCL-1 | | | | | | | |
| IL-11 | | 100.0% | 78.5% | 34.4% | 100.0% | 4.65 | 0 |
| and/or | | (84.5-100.0%) | (72.0-83.8%) | (23.7-47.0%) | (97.4-100.0%) | (3.53-6.12) | |
| CCL-1 | | | | | | | |

### CONCLUSION

Several non-invasive techniques have been studied for the detection of lung cancer. Imaging techniques, such as chest X-ray, low-dose spiral computerised tomography, sputum cytology and molecular biomarkers in different biological samples have been investigated to establish their diagnostic value for the early detection of lung cancer (Patz et al., 2010. J Thorac Oncol 5, 1502-1506; Hoffman et al, 2000, Lancet 355, 479-485). Although these tests vary in sensitivity and specificity, only low-dose chest computerised tomography has shown to reduce specific lung cancer mortality (Manser et al, 2003. Thorax 58, 784-789; Manser et al, 2004. Cochrane Database Syst Rev CD001991). In this invention protein markers involved in inflammation are located, which are involved in the pathogenesis of the two most common and devastating respiratory diseases associated with smokers: lung cancer and COPD.

In the validation study and using similar control groups, the ROC curves evidenced optimum cut-off levels for the diagnosis of BAL that were 42 pg/mL for IL-11 and 39.5 pg/mL for CCL-1. The diagnostic precision of adenocarcinoma was confirmed by each biomarker. There was even a positive correlation between BAL levels of IL-11 and CCL-1, the measurement of both proteins optimised the sensitivity and specificity of the diagnosis at levels of 90% and 89% respectively. Oddly enough, both proteins were similar predictive factors for adenocarcinoma, without the presence of concomitant COPD. The results of this invention strongly suggest inflammatory differences between the two histological subtypes (SCC and adenocarcinoma).

In addition, there is a correlation between high expression levels of IL-11 with cell proliferation, invasiveness, metastasis and poor prognosis.

CCL-1 acts as a potent chemoattractant of monocytes and lymphocytes and it is believed to play a significant role in inflammatory conditions (Harpel et al. 2002. Isr Med Assoc J. 4, 1025-1027). Recently the follow-up of multiple reactions, a high-performance liquid chromatography following the tandem mass spectrometry method, allows the validation of biomarkers. The functioning of these proteins in the differential diagnosis of pleural effusion (for instance, adenocarcinoma of the lung, mesothelioma and adenocarcinoma of other origins and non-tumoural) would be also of value.

This invention indicates that the determination of the CCL-1 and IL-11 levels of the BAL samples by a simple test, such as ELISA, could improve the diagnosis of adenocarcinoma of the lung in high-risk smokers, despite the presence or absence of COPD.

1-4. (canceled)

5. A method for determining that an individual has one of the following:
a) no COPD or lung cancer;
b) COPD;
c) adenocarcinoma,
d) COPD and adenocarcinoma; or
e) COPD and squamous carcinoma; the method comprising:
i) obtaining an isolated biological sample of the individual; and
ii) quantifying the expression level of at least one gene selected from the group consisting of: IGFBP1, MIP1β, CCL-1, MIG, PDGFAA, GDF-15 and EGF.

6. The method according to claim 5, further comprising:
iii) comparing the expression level of at least one gene determined in step (ii) with a reference level.

7. The method according to claim 5, wherein step (ii) also comprises quantifying the expression level of at least one gene selected from the group consisting of: IL-6sR, IL-1a, IL-11, EOTAXIN-2, TNFIR, TNFRII, IGFBP2, and MCP-1.

8. The method according to claim 5, wherein step (ii) is fully or partially automated.

9. The method according to claim 5, wherein the biological sample is bronchoalveolar fluid.

10. The method according to claim 5, wherein the quantitation is performed by immunoassay.

11. The method according to claim 10, wherein the immunoassay is an ELISA.

12. The method according to claim 6 further comprising determining that the individual has no COPD or lung cancer, when no expression of genes IGFBP1, MIPβ, CCL-1, MIG and PDGFAA is found.

13. The method according to claim 6 further comprising determining that the individual has adenocarcinoma, when an increase in the expression level of genes IL-11 and/or CCL-1 relative to a reference level is found.

14. The method according to claim 6 further comprising determining that the individual has COPD when the expression of MIG is found, and no expression of CCL-1 and IGFBP1 is found.

15. The method according to claim 6 further comprising determining that the individual has adenocarcinoma when the expression of CCL-1 is found, and when the expression level of MIP1β is found to be lower than 20 pg/mL.

16. The method according to claim 6 further comprising determining that the individual has squamous carcinoma when the expression of PDGFAA or MIP1β at any level is found, and no expression of CCL-1 is found.

17. The method according to claim 6 further comprising determining that the individual has COPD and adenocarcinoma when the expression of CCL-1 at any level is found, and the expression level of VEGF is found to be lower than 200 pg/mL.

18. A method according to claim 6 further comprising determining that the individual has COPD and squamous carcinoma when the expression level of GDF-15 and VEGF is found to be higher than 50 pg/mL and 200 pg/mL, respectively, and no expression of CCL-1 and EGF is found.

19-20. (canceled)

21. A kit for carrying out the method of claim 5, comprising:
elements necessary to quantify the expression level of at least one gene selected from the group consisting of: IGFBP1, MIP1β, CCL-1, MIG, PDGFAA, GDF-15, VEGF and EGF; and
instructions for use.

22. The kit according to claim 21, further comprising the elements necessary to quantify the expression level of at least one gene selected from the group consisting from: IL-6sR, IL-1a, IL-11, EOTAXIN-2, TNFRI, TNFRII, IGFBP2 and MCP1.

23. The kit according to claim 22 comprising the elements necessary to quantify the expression level of CCL-1 and/or IL-11.

24. The kit according to claim 21 comprising, anti-CCL-1 and anti-IL-11 antibodies.

25. The kit according to claim 21 comprising anti-IGFBP1, anti-MIP-1B, anti-CCL-1, anti-MIG, anti-PDGFAA, anti-GDF-15, anti-VEGF or anti-EGF antibodies.

26. The kit according to claim 25 comprising anti-IL-6sR, anti-IL-1a, anti-IL-11, anti-EOTAXIN-2, anti-TNFRI, anti-TNFRII, anti-IGFBP2 and anti-MCP1 antibodies.

27. (canceled)

28. A computer readable storage means comprising software instructions that direct a computer to perform the steps of the method according to claim 5.

29. The storage means according to claim 28, comprising the antibodies anti-IGFBP1, anti-MIP-1B, anti-CCL-1, anti-MIG, anti-PDGFAA, anti-GDF-15, anti-VEGF or anti-EGF.

30-32. (canceled)

33. A transmissible signal comprising software instructions that direct a computer to perform the steps of the method according to claim 5.


SEQUENCE LISTING (32 sequences)

SEQ ID NO: 1 - DNA, 5928 bp, Homo sapiens
ggcggtcccctgttctccccgctcaggtgcggcgctgtggcaggaagccaccccctcggtcggccggtgcgcggggctgttgcgccatccgctccggctttcgtaaccgcaccctgggacggcccagagacgctccagcgcgagttcctcaaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagctggctgggtggcaccagcagcctggaccctgtggatgataaaacacaaacgggctcagcaaaagatgcttctcactgccatgccagcttatctcaggggtgtgcggcctttggcttcacggaagagccttgcggaaggttctacgccaggggaaaatcagcctgctccagctgttcagctggttgaggtttcaaacctccctttccaaatgcccagcttaaaggggctagagtgaacttgggccactgtgaagagaaccatatcaagactctttggacactcacacggacactcaaaagctgggcaggttggtgggggcctcggtgtggagaagcggctggcagcccacccctcaacacctctgcacaagctgcaccctcaggcaggtgggatggatttccagccaaagcctcctccagccgccatgctcctggcccactgcatcgtttcatcttccaactcaaactcttaaaacccaagtgccttagcaaattctgtttttctaggcctggggacggcttttacttaaaccgccaaggctgggggaagaagctctctcctccctttcttccctacagttgaaaaacagctgagggtgagtgggtgaataatacagtatctcagggcctggtcgttttcaacagaattataattagttcctcattagcattttgctaaatgtgaatgatgatcctaggcatttgctgaatacagaggcaactgcattggctttgggttgcaggacctcaggtgagaagcagaggaaggagaggagaggggcacagggtctctaccatcccctgtagagtgggagctgagtgggggatcacagcctctgaaaaccaatgttctctcttctccacctcccacaaaggagagctagcagcagggagggcttctgccatttctgagatcaaaacggttttactgcagctttgtttgttgtcagctgaacctgggtaactagggaagataatattaaggaagacaatgtgaaaagaaaaatgagcctggcaagaatgtgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggtggcccacgcacttcgggaggtcgaggcaggaggatcacttgagtccagaagtttgagatcagcctgggcaatgtgataaaaccccatctctacaaaaagcataaaaattagccaagtgtggtagagtgtgcctgaagtcccagatacttggggggctgaggtgggaggatctcttgagcctgggaggtcaaggctgcagtgagccgagattgcaccactgcactccagcctgggtgacagagcaagtgagaccctgtctcaaaaaaagaaaaagaaaaagaaaaaatattttccctattagagaagagattgtggtttcattctgtattttgtttttgtcttaaaaagtggaaaaatagcctgcctcttctctactctagggaaaaaccagcgtgtgactactcccccaggtggttatggagagggtgtccggtccctgtcccagtgccgagaaggaagcctcccacgactgcccggcagggtcctagaaattccccaccctgaaagccctgagctttctgctatcaaagaggttttaaaaaaatcccatttaaaaaaaatcccttacctcggtgccttcctctttttatttagttccttgagttgattcagctctgcaagaattgaagcaggactaaatgtctagttgtaacaccatgattaaccacttcagctgacttttctgtccgagctttgaaaattcagtggtgttagtggttacccagttagctctcaagttatcagggtattccagagtggggatatgatttaaatcagccgtgtaaccatggacccaatatttaccagaccacaaaacttttctaatactctaccctcttagaaaaaccaccaccatcaccagacaggtgcgaaaggatgaaagtgaccatgttttgtttacggttttccaggtttaagctgttactgtcttcagtaagccgtgattttcattgctgggcttgtctgtagattttagaccctattgctgcttgaggcaactcatcttaggttggcaaaaaggcaggatggccgggcgcggtggctcacgcctgtaatcctagcactttgggaggccaaggtgggaggattgcttgagctcaggagtttgagaccaacctgggtaacatagtgagacaccatctctattatgaacaataacagttaagaaaaaaaaaggcaggcaggcggttatggtggttccctcccatcccaccacataaagtttctgagacttgagaacagcaaaatgctgttaaagggaaatattaagaatgagaatctgcagtaagggtgattctgtgcccacagttcttcaattctttataccgttttacccacatgtggtgttaccaaagccgggcagaaccatgctagcggaagatgtgaaatccagatagctcattattgccaagagctaggcagctttgatctccaaattgttattgctttcatttttattgtaatggaattgctttgttttgtttttttgtttttgtattgaagagggttgttttccctttatttttcataagctaatgtaaatgaagaaaaaatgtcttctctgggctgtaggcctggctcagcgtacacaggtatacatcctaagctctctatgttctctaatctgtggtgactgaacatgtgtctcaatgcacggggcatttctacctgtgtttctgcagcacccccactgccttgagtccccagcagtgctgttatttgcctaacacctgtagccatctgccacgcagccagacgtgaaacgctgagacagagaccatttaggttaaatacgacagcttatcctgctgggtggggaaagtaaaaaatatgctggttcaaggcctaaagtaaaatgatcaataatgtttgtagcattaatgaaatattttcaagaaatgtgtccaggggtagcactggctatgttgacgaggcctttggtaactcagagagctcttggccctgatggggacttgcccttacgctttctttatcaggctctgagttcacacggagcctctggcacttccctgctgtcttgggagaaaggaaactggttgccgcggcaggttgtggaatctgttgctggaaccaggctggaagcccacctggtagtgaacagggcccagtggggcaggctgggcatgttgtggtctatgggtttgtttcctggagaatgttcaggaatgtcttcccagctgctttggtgctgagctctattatctcacagcacgtccagaaggctaacccaggtggggaggatgctgacaccagctccaggtggagttggtggtcttaatttggagatgcaggggcaacctgtgaccctttgaggcaagagccctgcacccagctgtcccgtgcagccgtgggcaggggctgcacacggaggggcaggcgggccagttcagggtccgtgccaggccctcctcagtgccctgtgaaggcctcctgtcctccgtgcggctgggcaccagcaccagggagtttctatggcaaccttagtgattattaaggaacactgtcagttttatgaacatatgctcaaatgaaattctactttaggaggaaaggattggaacagcatgtcacaaggctgttaattaacagagagaccttattggatggagatcacatctgttaaatagaatacctcaactctacgttgttttcttggagataaataatagtttcaagtttttgtttgtttgttttacctaattacctgaaagcaaataccaaaggctgatgtctgtatatggggcaaagggtcagtatatttttcagtgtttttttttctaccagctattttgcatttaaagtgaacattgtgtttggaataaatactcttaaaaaataaaaaaaaaaaaaaa

SEQ ID NO: 2 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 3 - DNA, 2943 bp, Homo sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 4 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 5 - DNA, 2354 bp, Homo sapiens
gctcagggcacatgcctcccctccccaggccgcggcccagctgaccctcggggctcccccggcagcggacagggaagggttaaaggcccccggctccctgccccctgccctggggaacccctggccctgtggggacatgaactgtgtttgccgcctggtcctggtcgtgctgagcctgtggccagatacagctgtcgcccctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtgacccggggcccaaagccaccaccgtccttccaaagccagatcttatttatttatttatttcagtactgggggcgaaacagccaggtgatccccccgccattatctccccctagttagagacagtccttccgtgaggcctggggggcatctgtgccttatttatacttatttatttcaggagcaggggtgggaggcaggtggactcctgggtccccgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgtctgagagctggggctggatgcttgggtaactggggcagggcaggtggaggggagacctccattcaggtggaggtcccgagtgggcggggcagcgactgggagatgggtcggtcacccagacagctctgtggaggcagggtctgagccttgcctggggccccgcactgcatagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacgccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaat

SEQ ID NO: 6 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 7 - DNA, 542 bp, Homo sapiens
acagtggtgagctcttagcttcaccaggctcatcaaagctgctccaggaaggcccaagccagaccagaagacatgcagatcatcaccacagccctggtgtgcttgctgctagctgggatgtggccggaagatgtggacagcaagagcatgcaggtacccttctccagatgttgcttctcatttgcggagcaagagattcccctgagggcaatcctgtgttacagaaataccagctccatctgctccaatgagggcttaatattcaagctgaagagaggcaaagaggcctgcgccttggacacagttggatgggttcagaggcacagaaaaatgctgaggcactgcccgtcaaaaagaaaatgagcagatttctttccattgtgggctctggaaaccacatggcttcacctgtccccgaaactaccagccctacaccattccttctgccctgcttttgctaggtcacagaggatctgcttggtcttgataagctatgttgttgcactttaaacatttaaattatacaatcatcaacccccaac

SEQ ID NO: 8 - protein, 96 aa, Homo sapiens
MQIITTALVCLLLAGMWPEDVDSKSMQVPFSRCCFSFAEQEIPLRAILCYRNTSSICSNEGLIFKLKRGKEACALDTVGWVQRHRKMLRHCPSKRK

SEQ ID NO: 9 - DNA, 360 bp, Homosapiens
atggcaggcctgatgaccatagtaaccagccttctgttccttggtgtctgtgcccaccacatcatccctacgggctctgtggtcatcccctctccctgctgcatgttctttgtttccaagagaattcctgagaaccgagtggtcagctaccagctgtccagcaggagcacatgcctcaaggcaggagtgatcttcaccaccaagaagggccagcagttctgtggcgaccccaagcaggagtgggtccagaggtacatgaagaacctggacgccaagcagaagaaggcttcccctagggccagggcagtggctgtcaagggccctgtccagagatatcctggcaaccaaaccacctgctaa

SEQ ID NO: 10 - protein, 119 aa, Homo sapiens
MAGLMTIVTSLLFLGVCAHHIIPTGSVVIPSPCCMFFVSKRIPENRVVSYQLSSRSTCLKAGVIFTTKKGQQFCGDPKQEWVQRYMKNLDAKQKKASPRARAVAVKGPVQRYPGNQTTC

SEQ ID NO: 11 - DNA, 2809 bp, Homo sapiens
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgtccaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccgcgctgcgggcgcgcactccctgtttctcctcctcctggctggcgctgcctgcctctccgcactcactgctcgcgccgggcgcgctccgccagctccgtgctccccgcgccaccctcctccgggccgcgctccctaagggatggtactgaatttcgccgccacaggagaccggctggagcgcccgccccgcggcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccatgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacgggaaggcctagggagtcaggtaaaaaacggaaaagaaaaaggttaaaacccacctaaagcagccaaccagatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagaaagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcaggggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgtttttttttttttttttttacaccataaagtgattattaagctttcctttttactctttggctagctttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttctttttttgttgatccaaaattaaaaaaaaaaataccaccaacaaaa

SEQ ID NO: 12 - protein, 211 aa, Homo sapiens [sequence omitted]

SEQ ID NO: 13 - DNA, 2258 bp, Homo sapiens
ctcctccagctcttcctgtcccgctgttgcaacactgcctcactcttcccctcccaccttctctcccctcctctctgctttaattttctcagaattctctggactgaggctccagttctggcctttggggttcaagatcactgggaccaggccgtgatctctatgcccgagtctcaaccctcaactgtcaccccaaggcacttgggacgtcctggacagaccgagtcccgggaagccccagcactgccgctgccacactgccctgagcccaaatgggggagtgagaggccatagctgtctggcatgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcagatgaggctgcgcccctgcgggcagctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggccttcagctggagctgtggacttttgtacatacactaaaattctgaagttaaagctctgctcttggaaaaaaaaaaaaaaaaaaaaaaaaaaaaaaaaa

SEQ ID NO: 14 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 15 - DNA, 3682 bp, Homo sapiens
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 16 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 17 - DNA, 5600 bp, Homo sapiens
aaaaagagaaactgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggagcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgcaaaatgagttgtgatgaatcaatgaaaaatgtaatttagaaactgatttcttcagaattagatggcttattttttaaaatatttgaatgaaaacattttatttttaaaatattacacaggaggcttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacataaacattttattgtatgcctgattaagtagttaattatagtctaaggcagtactagagttgaaccaaaatgatttgtcaagcttgctgatgtttctgtttttcgttttttttttttttccggagagaggataggatctcactctgttatccaggctggagtgtgcaatggcacaatcatagctcagtgcagcctcaaactcctgggctcaagcaatcctcctgcctcagcctcccgagtaactaggaccacaggcacaggccaccatgcctggctaaggtttttatttttattttttgtagacatggggatcacacaatgttgcccaggctggtcttgaactcctggcctcaagcaaggtcgtgctggtaattttgcaaaatgaattgtgattgactttcagcctcccaacgtattagattataggcattagccatggtgcccagccttgtaacttttaaaaaaattttttaatctacaactctgtagattaaaatttcacatggtgttctaattaaatatttttcttgcagccaagatattgttactacagataacacaacctgatatggtaactttaaattttgggggctttgaatcattcagtttatgcattaactagtccctttgtttatctttcatttctcaaccccttgtactttggtgataccagacatcagaataaaaagaaattgaagtaaaaaaaaaaaaaaaaaa

SEQ ID NO: 18 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 19 - DNA, 667 bp, Homo sapiens
agcacaggacacagctgggttctgaagcttctgagttctgcagcctcacctctgagaaaacctctttgccaccaataccatgaagctctgcgtgactgtcctgtctctcctcatgctagtagctgccttctgctctccagcgctctcagcaccaatgggctcagaccctcccaccgcctgctgcttttcttacactgcgaggaagcttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaactgagctgctcagagacaggaagtcttcagggaaggtcacctgagcccggatgcttctccatgagacacatctcctccatactcaggactcctctccgcagttcctgtcccttctcttaatttaatcttttttatgtgccgtgttattgtattaggtgtcatttccattatttatattagtttagccaaaggataagtgtcccctatggggatggtccactgtcactgtttctctgctgttgcaaatacatggataacacatttgattctgtgtgttttcataataaaactttaaaataaaatgcagacagtt

SEQ ID NO: 20 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 21 - DNA, 2545 bp, Homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 22 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 23 - DNA, 760 bp, Homo sapiens
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 24 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 25 - DNA, 1439 bp, Homo sapiens
tgcggcggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaa

SEQ ID NO: 26 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 27 - DNA, 1660 bp, Homo sapiens
ggtgcactagcaaaacaaacttattttgaacactcagctcctagcgtgcggcgctgccaatcattaacctcctggtgcaagtggcgcggcctgtgccctttataaggtgcgcgctgtgtccagcgagcatcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtctataatggaaacatttacaataaatattctgcatggaaatactgttaaaaaaaaaaa

SEQ ID NO: 28 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 29 - DNA, 1220 bp, Homo sapiens
agtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcggaggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttaaaaaaaaaaaaaaaaaaaa

SEQ ID NO: 30 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 31 - DNA, 3677 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 32 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR